SEQUENCE LISTING (48 sequences)

SEQ ID NO: 1 - RNA, 25 nt, artificial
  siRNA molecule
ugggcuccgucaguuagauuguuaa

SEQ ID NO: 2 - RNA, 25 nt, artificial
  siRNA molecule
uuaacaaucuaacugacggagccca

SEQ ID NO: 3 - RNA, 25 nt, artificial
  siRNA molecule
cacauaucuacaggaggauauggua

SEQ ID NO: 4 - RNA, 25 nt, artificial
  siRNA molecule
uaccauauccuccuguagauaugug

SEQ ID NO: 5 - RNA, 25 nt, artificial
  siRNA molecule
ccuaaagauggagguaugaugcaaa

SEQ ID NO: 6 - RNA, 25 nt, artificial
  siRNA molecule
uuugcaucauaccuccaucuuuagg

SEQ ID NO: 7 - RNA, 25 nt, artificial
  siRNA molecule
uggcucuauauagcccuccucuaau

SEQ ID NO: 8 - RNA, 25 nt, artificial
  siRNA molecule
auuagaggagggcuauauagagcca

SEQ ID NO: 9 - RNA, 25 nt, artificial
  siRNA molecule
cgaccgaguuaaaggaaacuuuguu

SEQ ID NO: 10 - RNA, 25 nt, artificial
  siRNA molecule
aacaaaguuuccuuuaacucggucg

SEQ ID NO: 11 - RNA, 25 nt, artificial
  siRNA molecule
gcaacggaaucggacgcaaucagaa

SEQ ID NO: 12 - RNA, 25 nt, artificial
  siRNA molecule
uucugauugcguccgauuccguugc

SEQ ID NO: 13 - RNA, 25 nt, artificial
  siRNA molecule
cggacauauuaggaguucuuacuau

SEQ ID NO: 14 - RNA, 25 nt, artificial
  siRNA molecule
auaguaagaacuccuaauauguccg

SEQ ID NO: 15 - RNA, 25 nt, artificial
  siRNA molecule
uaguggauccgacguuucaacuauu

SEQ ID NO: 16 - RNA, 25 nt, artificial
  siRNA molecule
aauaguugaaacgucggauccacua

SEQ ID NO: 17 - RNA, 25 nt, artificial
  siRNA molecule
ccucaugagcugacguuagacauaa

SEQ ID NO: 18 - RNA, 25 nt, artificial
  siRNA molecule
uuaugucuaacgucagcucaugagg

SEQ ID NO: 19 - RNA, 25 nt, artificial
  siRNA molecule
cggcuauuagagguaaugauguuau

SEQ ID NO: 20 - RNA, 25 nt, artificial
  siRNA molecule
auaacaucauuaccucuaauagccg

SEQ ID NO: 21 - RNA, 25 nt, artificial
  siRNA molecule
uagggaugaggaagcauacucuaua

SEQ ID NO: 22 - RNA, 25 nt, artificial
  siRNA molecule
uauagaguaugcuuccucaucccua

SEQ ID NO: 23 - RNA, 25 nt, artificial
  siRNA molecule
caaguagccaauggcgccauagauu

SEQ ID NO: 24 - RNA, 25 nt, artificial
  siRNA molecule
aaucuauggcgccauuggcuacuug

SEQ ID NO: 25 - RNA, 25 nt, artificial
  siRNA molecule
gagccauugcgagcaugauaaugua

SEQ ID NO: 26 - RNA, 25 nt, artificial
  siRNA molecule
uacauuaucaugcucgcaauggcuc

SEQ ID NO: 27 - RNA, 25 nt, artificial
  siRNA molecule
caucgccuacaaaugacacugauaa

SEQ ID NO: 28 - RNA, 25 nt, artificial
  siRNA molecule
uuaucagugucauuuguaggcgaug

SEQ ID NO: 29 - RNA, 25 nt, artificial
  siRNA molecule
acucgaacccucauuggcuacauuu

SEQ ID NO: 30 - RNA, 25 nt, artificial
  siRNA molecule
aaauguagccaaugaggguucgagu

SEQ ID NO: 31 - RNA, 25 nt, artificial
  siRNA molecule
ccucaucgaagaagauaccauauuu

SEQ ID NO: 32 - RNA, 25 nt, artificial
  siRNA molecule
aaauaugguaucuucuucgaugagg

SEQ ID NO: 33 - RNA, 25 nt, artificial
  siRNA molecule
ccgaguaugauauacuccauguuga

SEQ ID NO: 34 - RNA, 25 nt, artificial
  siRNA molecule
ucaacauggaguauaucauacucgg

SEQ ID NO: 35 - RNA, 25 nt, artificial
  siRNA molecule
ccacagguaauuaugugacuguuga

SEQ ID NO: 36 - RNA, 25 nt, artificial
  siRNA molecule
ucaacagucacauaauuaccugugg

SEQ ID NO: 37 - DNA, 1320 bp, Vaccinia virus
ttagtttccgccatttatccagtctgagaaaatgtctctcataataaatttttccaagaaactaattgggtgaagaatggaaacctttaatctatatttatcacagtctgttttggtacacatgatgaattcttctaatgctgtactaaattcgatatctttttcgatttctggatatgtttttaataaagtatgaacaaagaaatggaaatcgtaataccagttatgttcaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcaacagggtagttcgtagattttaatgtataacagagccatctgtaaaaagataaactttatgtattgtaccaaagatttaaatcctaatttgatagctaactcggtatctactttatctgccgaatacagtgctaggggaaaaattataatatttcctctttcgtattcgtagttagttctcttttcatgttcgaaaaagtgaaacatgcggttaaaatagtttataacattaatattactgttaataactgccggataaaagtgggatagtaatttcacgaatttgatactgtcctttctctcgttaaacgcctttaaaaaaactttagaagaatatctcaatgagagttcctgaccatccatagtttgtatcaataatagcaacatatgaagaacccgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccttattaaaattcttaaaataatttaacaatctaactgacggagcccaattttggtgtaaatctaattgggacattatgttgttaaaatacaaacagtctcctaatataacagtatctgataatctatggggagacatccattgatattcaggggatgaatcattggcaacacccat

SEQ ID NO: 38 - DNA, 612 bp, Vaccinia virus
atgagttatttaagatattacaatatgcttgacgacttctctgcgggtgctggagtgcttgataaagatttatttacagaggaacagcagcaatcgtttatgcctaaagatggaggtatgatgcaaaacgattatggaggaatgaatgattatttgggaatcttcaaaaataatgatgttagaacgttactcggtttgattttgttcgtcttggctctatatagccctcctctaatctctatattgatgatatttatctcatcttttctattgcctcttactagcttagttattacctattgcttagtaactcaaatgtatcgtggaggtaatggcaacactgtgggaatgtctattgtgtgtattgtagctgctgtaattattatggcaatcaatgtatttacgaattcacagatatttaatattatttcttacattattttgtttattctgttctttgcatatgtgatgaacatcgaaagacaggactatagaagaagtataaatgtaaccattcctgaacagtatacctgcaacaaaccttatactgcgggaaataaggtagatgttgatataccaacatttaacagtttaaatactgacgattattaa

SEQ ID NO: 39 - DNA, 852 bp, Vaccinia virus
atgcagcatccgcgggaagagaattcaatcgtcgttgaactcgaaccctcattggctacatttatcaaacaaggatttaataatctcgtaaaatggcccttgttaaacattggaatagttttgtctaatacatctaccgctgtcaatgaggaatggctaactgcggtagagcatattcccaccatgaagatattttacaaacatatacataagatacttactagagaaatggggtttttagtctatttgaaaagatcccaatctgaacgcgataattatataactttatacgattttgattattatattatagataaggatacaaattctgtaactatggtagataaaccgaccgagttaaaggaaactttgttacatgtatttcaagaatatcgtttaaagagttctcaaacaatagagcttatagcgtttagttcaggtacggtaataaacgaagacatagtttcaaaattaacatttttagatgtggaggtatttaatagagaatataataatgttaaaactatcatagatccggattttgtatttagatctccatttatagttatttctcctatgggtaaactaactttcttcgtagaagtatattcgtggtttgattttaaatcgtgtttcaaagatattatagatttcttagaaggtgctctaatagccaatattcataatcacatgattaaggtaggtaattgtgacgaaacagtatcgtcttataatccagagtctggaatgttgtttgttaatgacttaatgactatgaacatagtcaactttttcggatgtaattctaggttagaatcataccatcggttcgatatgacaaaagtagatgtt

SEQ ID NO: 40 - DNA, 1776 bp, Vaccinia virus
atgattgtcttaccgaataaagttcgtattttcatcaacgatcggatgaaaaaggatatctacttgggaatttctaatttcggattcgagaatgatatagatgaaatcttgggaattgctcacttgttggaacatctacttatatcctttgattctactaattttttagcgaatgcttctacatctagaagttatatgagtttttggtgtaaatccattaattcagcaacggaatcggacgcaatcagaacattagtttcgtggttcttttctaacggaaaactcaaagataatttttccctttctagtatacgatttcacattaaagaattagaaaacgaatactattttagaaatgaagtattccattgtatggatatactaacgtttcttagcggaggcgatttatataacggtgggagaatagacatgatagataatcttaatatagttcgtgatatgctggtaaatagaatgcaaaggatatcgggatcgaatatcgtaatttttgttaagagattaggacctggaacattggatttcttcaaacagacatttgggtctttaccagcatgtccggagattattccttcgtctattccagtaagtacaaacggtaaaatagttatgactccgtctccattttatacagttatggtaaagattaatccaacattagataatattttagggattctgtatttgtacgaaacttaccacttaatagactatgagactatcggcaaccagttatatttaacggtatcctttatcgatgaaactgaatacgagagctttcttcgtggcgaggctatattacaaattagtcaatgtcaacgtattaatatgaattatagcgacgattatatgatgaacatctatttgaattttccttggctatcgcatgatttatatgattacattacacgtattaatgacgatagcaagtcgatactaatatccttgacgaatgaaatatatgcatctataattaatagagatatcatagttatttacccaaactttagtaaggccatgtgtaacactagagatacccaacaacatccgatagtagttcttgacgcaaccaatgatggactgattaagaaaccttatagaagtatacccctaatgaagcgtctaacatctaatgaaatatttatacgatacggagacgcgtctctcatggacatgataactttatcattgtctaaacaagatatatcattaaaaagaaatgccgaaggaatacgtgtaaaacatagtttttcagctgatgatatacaggcaattatggaatctgattcgtttttaaagtatagtagatcaaaaccagctgcgatgtatcaatatatatttctatcattttttgctagtggtaattccatagatgacatattggcaaatagagattctaccttagaattttctaaaagaactaaaagtaaaattttgtttggtaggaataccaggtacgacgtcactgcaaaatctagttttgtatgtggtatagtacgaggtaaatcattggataaaacgtctctggttgaaatgatgtgggatctcaagaagaaaggattaatatattctatggaatttaccaatctattgagtaagaataccttttatttgttcacatttactatctacactgatgaagtatacgattatctaaacactaataaacttttttttgcaaaatgtttagtcgtgtctacaaaaggagatgtagaaaatttttcatctctaaaaaaagatgtggtcattagagtttga

SEQ ID NO: 41 - DNA, 1440 bp, Vaccinia virus
atgaataggaatcctgatcagaatactcttcctaatattacattaaagattatagaaacctatttaggcagagtacctagtgtgaacgaatatcatatgttaaaattacaagctagaaatattcaaaaaataactgtttttaacaaagacatatttgtatctttagtaaaaaagaataaaaaaagatttttttccgatgttaatacatctgcatcagaaataaaagatcgtatacttagctacttttctaaacagactcaaacatataatataggtaaattatttacgattatagaactacaatctgtattagtgaccacatacacggacatattaggagttcttactattaaagctccaaatgtaatttcatctaaaatttcttataatgtaacatcaatggaagaattggcaagagatatgctaaattctatgaacgtcgcagtaatagacaaggcaaaagtaatgggacgtcataatgtatcttccctagtcaaaaatgttaataagttgatggaagaatatcttagacgccataataaaagttgtatatgttacggatcatattctctatatctaattaatccaaatatacggtacggcgatatagatattcttcagactaattctagaacttttcttatagatttggcctttctaataaaatttatcacgggaaataatattatattaagtaaaatcccatatcttagaaactatatggtgataaaagatgaaaacgataatcatatcattgatagttttaatattcgccaggataccatgaacgtagttcctaaaatctttatagataatatctatatagtggatccgacgtttcaactattgaacatgataaaaatgttttctcaaatagatagattggaagatctatccaaagatcctgaaaagtttaatgcgcgtatggcaaccatgctagaatacgttagatatacacatggtatagtctttgatggtaagcgtaataatatgccgatgaaatgtatcatcgatgaaaataatcgcatagttactgtcactactaaagactattttagctttaaaaaatgtctagtgtatctagatgaaaatgtgttatcgagtgatatattagatcttaacgccgacacatcgtgtgatttcgagagtgttacaaattctgtatatctaattcatgataatatcatgtatacatatttctcaaatactattctccttagtgataaggggaaggtacatgaaataagtgccagaggtttatgtgcacatatattgttgtatcagatgctgacatctggagaatacaaacaatgtttatcggatctcttaaattcgatgatgaatagagataaaatacctatctattcacatactgaaagagataaaaaacctggacgacacggatttattaatatcgaaaaggatataattgtattttag

SEQ ID NO: 42 - DNA, 486 bp, Vaccinia virus
atgtcgagctttgttaccaatggataccttccagttacattggaaccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgtcgactcataa

SEQ ID NO: 43 - DNA, 2358 bp, Vaccinia virus
atggatgcggctattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggcgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaacaagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacaacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagaccatatagtttggattaataattcatggaaatttaacagcgaagaacccttgataacaaaactaattttgtcaataagacatcaactacctaaggaatattcaagcgaattactctgtccaagaaaacgaaagactgtagaagctaacatacgagacatgttagtagattcagtagagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagctatatgaaaaaacattatctagttgtttatgcggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacacccgaagagattcctgactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaa

SEQ ID NO: 44 - DNA, 327 bp, Vaccinia virus
atgtcatgttatacagctatattaaaatctgtaggaggactggcgctatttcaagtagccaatggcgccatagatttatgtagacatttctttatgtatttttgtgaacaaaagctacgaccaaattcattttggttcgttgttgttagagccattgcgagcatgataatgtatttagtattaggtatagcattgctgtatatttctgaacaagatgacaagaagaatactaataatgccaacactaataatgatagtaatagtaataatagtaacaatgataaacgaaatgagtcgtctataaattctaactccagtcctaagtaa

SEQ ID NO: 45 - DNA, 945 bp, Vaccinia virus
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccggaaactagttctaagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagatcatacagttacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaattttctgtattacatattatatatataataaacgttcacgtaaatacaaaacagagaacaaagtctag

SEQ ID NO: 46 - DNA, 756 bp, Vaccinia virus
atgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgaaaatatcacaggagcaatgtttaccacaatgtataatataagaaacaatttgggtctaggagatgataaactaactattgaagccattgaaaactatttcttggatcctaacaatgaagttatgcctcttattattaataatacggatatgactgccgtcattcctaaaaaaagtggtaggagaaagaataagaacatggttatcttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatatcctaaatgtgtattctccttccacatccataagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattga

SEQ ID NO: 47 - DNA, 327 bp, Vaccinia virus
atggccgaggaatttgtacaacaaaggttggccaataacaaagtgacaatttttgtcaagtatacatgtcctttttgtagaaatgcactggatattctaaataagtttagtttcaaaagaggagcgtatgaaattgtcgatattaaagaatttaaacccgaaaatgaattgcgtgactattttgaacaaattactggtggtagaactgttcctagaatcttttttgggaaaacttctattggtggatatagcgacctgttggaaatagacaacatggacgcattgggtgatattctatcatctattggggtattgagaacttgttga

SEQ ID NO: 48 - DNA, 3021 bp, Vaccinia virus
atggatgttcggtgcattaattggtttgaaagtcacggtgaaaacagatttttatatctgaaatccagatgtcgaaatggtgagaccgtatttatacgatttcctcattacttttattacgtagtgacggacgaaatatatcagtcattgtctcctcctccatttaatgcgaggccgttgggaaagatgagaactatagacattgacgagacaataagttataatctagatattaaagatagaaaatgctccgtcgcagatatgtggttgatagaagagccaaagaaacgcagcatacaaaatgccaccatggatgaatttctcaatattagttggttttatatttctaacgggatatctccagacggatgttactcgttggacgagcaatatttgacaaagattaacaatggatgttatcattgtgacgatccacgtaactgtttcgctaaaaaaatacctagattcgatatcccaagatcgtacttatttctagatatagagtgtcacttcgataagaagtttccttctgtatttattaacccaatctcgcatacaagttactgttatatcgatttaagtggtaaacgattattgtttacgctcattaatgaagagatgttaacggaacaggaaatacaagaagccgtcgatagaggatgtttgaggatacagtcactaatggaaatggattacgaacgagaactagttttatgttctgaaatagttttgttacgaatagctaaacaattgttggaactaacgttcgactatgtcgttacctttaacggacataactttgatctgagatatattactaatcgtctagagttattaacaggagagaagattatctttagatctccggacaaaaaggaagctgtacatctctgtatttatgagagaaatcagtctagtcataagggagtaggcggcatggccaatactacgtttcacgttaataacaataatggaactatatttttcgatctatattcattcattcaaaaatctgaaaaattggattcgtacaaattggattctatatccaagaacgcgttcagttgcatgggtaaagtattaaatagaggagttagagaaatgacgttcatcggtgacgatactacggacgcgaaaggcaaagccgctgcatttgcaaaggttttaaccacaggtaattatgtgactgttgatgaggatattatatgtaaagtaattcgtaaagatatttgggaaaatggatttaaagtcgtactattatgtcctactttacctaatgatacatataaattatctttcggaaaggatgacgttgatttagctcagatgtataaggattataatctaaacatagctttagatatggctagatactgtattcatgatgcttgtttgtgtcagtatttgtgggagtattatggagtagaaacaaaaacagacgcgggtgcgtcaacatatgtgcttcctcaatccatggtattcgaatatagagcgagtacagtcatcaagggtccactgttaaagctattgttggaaactaaaactatcttagttagatcagaaacaaaacaaaagtttccttatgaaggcggtaaggtatttgctccaaaacaaaaaatgtttagtaataatgtattaatctttgattataacagtctgtatcctaatgtgtgtatctttggaaatctatctccggaaacattagtcggtgtcgttgttagtaccaatagattggaagaagaaataaataatcagctcttgcttcagaaatatccacctcctagatatattacggttcattgtgaacctagactaccgaacctcatctctgaaatagcaattttcgatagatcgatagaaggaaccattcctagactattaagaacatttttggcagagagagccagatataaaaagatgttaaaacaggctaccagttcaactgaaaaggccatctatgattccatgcaatatacgtacaagatagtagccaactcagtatatggtctgatgggatttagaaatagtgctctatactcatacgcttcggctaagagttgcacatccataggacgtagaatgatcttgtatctagaatcggtactaaatggagcagagttatctaacggtatgttacggtttgccaatccattaagtaatccattttatatggacgatagagatattaatccgattgtgaaaacatcgttgcctatagattacagatttcgttttcgtagcgtgtatggagataccgactccgtgtttacagagatagacagtcaagatgtagataagtccatagaaatagcaaaggagttagaacgactgattaataatagagtattgtttaataattttaaaatagagtttgaggcggtatataagaatctgattatgcaatcgaagaagaaatatacaacgatgaaatactcggcatcgtcgaattcaaaatctgtacctgagagaattaataaaggtactagtgaaactagaagagatgtttccaagtttcataagaatatgattaagacatacaagaccagactgtctgagatgttgtctgaaggacggatgaattctaatcaggtatgtatagatattctccgttctttagaaacagatttacgatccgaatttgatagtagatcgtctcctctagaattatttatgttgagtcgaatgcatcactcaaattataaatccgcagataaccctaatatgtatttggttactgaatataataaaaataatccagaaactatagaacttggagaacgatattattttgcatatatttgtccggctaatgtaccatggaccaaaaaacttgtaaatattaaaacatatgaaacaattatcgatagaagttttaaactcggcagtgatcaaagaatattttacgaagtttactttaaacgattgacgtccgaaatagtcaatctattggataataaagttttatgcatctcattctttgaaagaatgtttggttcaaaacctacattttacgaagcataa

(19) United States
(12) Patent Application Publication
(10) Pub. No.: US 2010/0204300 A1
(43) Pub. Date: Aug. 12, 2010

(54) ANTI-VIRAL METHODS AND COMPOSITIONS

(76) Inventors: Stephen Mark Tompkins, Watkinsville, GA (US); Jeffrey Robert Hogan, Athens, GA (US)

Correspondence Address: MUETING, RAASCH & GEBHARDT, P.A., P.O. BOX 581336, MINNEAPOLIS, MN, 55458-1336, US

(21) Appl. No.: 12/522,456

(22) PCT Filed: Jan. 16, 2008
(86) PCT No.: PCT/US08/51203

§ 371 Date: Oct. 6, 2009

Related U.S. Application Data
(60) Provisional application No. 60/880,930, filed on Jan. 17, 2007.

(51) Int. Cl. A61K 31/7105; C07H 21/02; C12N 15/74; C12N 5/10; A61P 31/12

(52) U.S. Cl. 514/44 A; 536/24.5; 435/320.1; 435/325

(57) ABSTRACT

The instant disclosure relates to methods and compositions for silencing poxvirus gene expression and replication using RNA interference. In certain embodiments, the disclosure relates to methods of treating a subject with a poxvirus infection.

### RELATED APPLICATIONS

This application claims the benefit of priority of U.S. Provisional Patent Application Ser. No. 60/880,930, filed Jan. 17, 2007, the content of which is incorporated by reference in its entirety.

### FIELD

Aspects and embodiments of the instant disclosure relate generally to therapeutic methods and compositions associated with poxvirus gene modulation. In certain aspects, the instant disclosure is directed to silencing Poxvirus gene expression and inhibiting poxvirus replication by RNA interference

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

RNA interference (RNAi) is an evolutionarily conserved, gene-silencing mechanism wherein small double-stranded RNA molecules, or small interfering RNA (siRNA), targets cognate RNA for destruction with exquisite potency and selectivity causing post-transcriptional gene silencing. The RNAi machinery, which is expressed in all eukaryotic cells, has been shown to regulate the expression of key genes involved in cell differentiation in plants and animals. Given the power of RNAi to silence genes, numerous RNAi based therapeutic strategies are being developed, particularly for RNA viruses such as HIV, influenza and respiratory syncytial virus. Development of synthetic siRNA drugs is particularly useful in situations in which long-term silencing is not required or undesirable, e.g. treating acute viral infections.

Homology-dependent gene silencing was discovered in transgenic plants in the form of co-suppression between introduced transgenes or between a transgene and its homologous endogenous gene. Both RNA silencing and RNAi are generic terms to describe gene silencing mechanisms guided by siRNAs and microRNAs (miRNA). The central feature of RNA silencing is the production of siRNAs by the endoribonuclease, Dicer. siRNAs are asymmetrically assembled into effector complexes called RNA-induced silencing complexes (RISC). siRNAs control the specificity of RNA silencing by recruiting the effector complex to a cognate single-stranded RNA target, leading to either slicing or translational arrest of nascent RNA synthesis.

Synthetic siRNAs delivered to a cell are incorporated into RISC complexes. Within the RISC complex, the two strands of the siRNA become separated, so that they can target complementary sequences in mRNAs. After pairing with an siRNA strand, the targeted mRNA is precisely cleaved and undergoes degradation thereby interrupting the synthesis of the targeted protein. The RISC complex is naturally stable within the cell, and once formed, will continue to seek and destroy the targeted mRNA molecules, resulting in sustained suppression of specific protein transcript synthesis.

Poxviruses have been studied for more than 200 years. From the time of Edward Jenner's pioneering vaccination experiments in 1796 to the present, poxviruses that infect nearly every vertebrate animal on the planet, from crocodiles to humans, have been identified. In addition, smallpox was the first disease to be eradicated by man.

While nearly 30 years have passed since smallpox was declared eradicated, poxviruses continue to plague both animals and man as a zoonotic pathogen. For example, in 2003, 71 people in the United States became infected with monkeypoxvirus after interaction with prairie dogs. Based on analyses of sera for poxvirus-specific antibodies, a wide variety of animals including rats, rabbits, prairie dogs, and squirrels may also be potential reservoirs of poxviruses. Given the potential impact on both animals of agricultural importance and human health, new treatments for acute poxvirus disease are needed.

As a result of the termination of the smallpox vaccination program in 1972, it is estimated (based on U.S. government census data) that approximately 42% of the current U.S. population has no immunity to poxviruses. Furthermore, to date, there are no antiviral drugs approved by the F.D.A. or U.S.D.A. for the treatment of poxvirus infection in humans or animals.

### SUMMARY OF THE INVENTION

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

Accordingly, the instant disclosure is directed to the development of RNA interference (RNAi) technology as a therapeutic approach to reduce and/or inhibit viral replication or infection. In particular, aspects and embodiments of the present disclosure include methods and reagents for reducing and/or inhibiting Poxvirus infection and/or replication. In certain embodiments, the methods can employ, and the reagents can include, nucleic acid or polynucleotide molecules that bind to cognate poxvirus RNA. In a preferred embodiment, the nucleic acid molecule is double stranded.

In an embodiment, the present method can inhibit Poxvirus infection or replication. Inhibition can be achieved by administering a therapeutically effective amount of a nucleic acid polynucleotide molecule.

### BRIEF DESCRIPTION OF THE FIGURES

FIG. 1 shows the summary of screening results. At least 87 individual siRNAs were designed that targeted at least 30 different poxvirus genes. Each siRNA was tested at least 2 times to inhibit replication of vaccinia virus replications in vitro. Replication of vaccinia virus in the cells transfected with each siRNA was compared to replication of vaccinia virus in the cells transfected with green fluorescent protein (GFP)-specific or influenza nucleoprotein (fluNP)-specific siRNAs and a percent inhibition calculated and graphed.

FIG. 2: Poxvirus gene-specific siRNA molecules blocked virus replication in vitro. Vero E6 cells were grown in 6 well plates and transfected with siRNA molecules at 100 nM using Lipofectamine™ 2000 (Invitrogen, Carlsbad, Calif.). After approximately 16 hours, the cells were infected with cowpoxvirus (Brighton strain) at a multiplicity of infection (MOI) of approximately 0.2 plaque forming units (PFU)/cell. At 48 hours post-infection, the cells were fixed with methanol and stained with crystal violet to visualize plaques. * indicates treatments that were significant from the influenza control, while ‡ indicates the treatments that are significant from all other treatments. Differences were analyzed using post hoc one way ANOVA (pairwise, Holm-Sidak method) with alpha=0.05.

FIG. 3 shows confirmation of siRNA-specific reduction of poxvirus replication as measured by classical plaque assay. Poxvirus gene-specific siRNA molecules block virus replication in vitro. Vero E6 cells were grown in 6 well plates and transfected with siRNA molecule at 100 nM using Lipofectamine 2000. After approximately 16 hours, the cells were infected with cowpox virus (Brighton Strain) at multiplicity of infection (MOI) of approximately 0.2 plaque forming units (PFU)/cell. At 48 hours post-infection, the cells were fixed with methanol and stained with crystal violet to visualize plaques.

FIG. 4 shows reduction in D5R gene expression in cells transfected with D5R-specific or control siRNA and infected with vaccinia virus. Gene expression was measured by real-time, RT-PCR and the data demonstrate greater than 227-fold reduction in D5R mRNA in D5R-specific siRNA treated cells. () Control siRNA treated cells. (▪) D5R-specific siRNA treated cells.

FIG. 5 also shows reduction in D5R gene expression in cells transfected with D5R-specific or control siRNA and infected with vaccinia virus. Gene expression was measured by real-time, RT-PCR. The control cells had 100% D5R mRNA expression, but the D5R-specific siRNA treated cells only had 0.44% D5R mRNA expression. The data demonstrate greater than 227-fold reduction in D5R mRNA in D5R-specific siRNA treated cells.

### DETAILED DESCRIPTION

The instant disclosure relates to compounds, compositions, and methods useful for modulating gene expression using short interfering nucleic acid (siNA) molecules. The disclosure also relates to compounds, compositions, and methods of treatment useful for modulating the expression and/or activity of viral genes and or proteins in a subject by RNA interference (RNAi) using small nucleic acid molecules. In particular, the instant disclosure include small nucleic acid polynucleotide molecules (e.g. 19-27 nucleotides), short interfering nucleic acid (siNA), short interfering RNA (siRNA), double-stranded RNA (dsRNA), micro-RNA (miRNA), short hairpin RNA (shRNA), and antisense polynucleotide molecules and methods of use thereof to modulate the expression of genes associated with for viral replication or infection.

### Definitions

As used herein, “subject” may include any mammals, including humans, domestic and farm animals, and zoo, sports, or pet animals, such as dogs, horses, cats, sheep, pigs, cows, etc. The preferred mammal herein is a human, including adults, children, and the elderly.

As used herein, “preventing” means preventing in whole or in part, or ameliorating or controlling.

As used herein, the term “treating” or “treatment” or “alleviation” refers to both therapeutic treatment and prophylactic or preventative measures and “wherein the object is to prevent or slow down (lessen) the targeted pathologic condition or disorder. Those in need of treatment include those already with a viral infection as well as those prone to having an infection or those in which an infection is to be prevented.

As used herein, a “therapeutically effective amount” or “effective amount” in reference to the polynucleotides or compositions of the instant disclosure refers to the amount sufficient to induce a desired biological, pharmaceutical, or therapeutic result. The result can be alleviation of the signs, symptoms, or causes of a disease or disorder or condition, or any other desired alteration of a biological system. In certain embodiments, the result will involve preventing, retarding, or reducing the incidence or severity of and/or decreasing viral infection and/or viral replication in whole or in part. Generally, alleviation or treatment of a disease or disorder involves the lessening of one or more symptoms or medical problems associated with the disease or disorder.

As used herein, the phrase “duplex region” refers to the region in two complementary or substantially complementary polynucleotides that form base pairs with one another, either by Watson-Crick base pairing or any other manner that allows for a duplex between polynucleotide strands that are complementary or substantially complementary. For example, a polynucleotide strand having 25 nucleotide units can base pair with another polynucleotide of 25 nucleotide units, yet only 23 bases on each strand are complementary or substantially complementary, such that the “duplex region” consists of 23 base pairs. The remaining base pairs may, for example, exist as 5′ and 3′ overhangs. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well know in the art. Alternatively, two strands can be synthesized and added together under biological conditions to determine if they anneal to one another.

As used herein, an siRNA having a sequence “sufficiently complementary” to a target mRNA sequence means that the siRNA has a sequence sufficient to trigger the destruction of the target mRNA by the RNAi machinery (e.g., the RISC complex) or process. The siRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand.

As used herein, “operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

Anti-Viral Polynucleotides and Agents

Exemplary anti-viral agents include agents that decrease or inhibit expression or function of viral genes/proteins associated with replication and/or infection. Anti-viral agents include anti-viral polynucleotides, such as, for example, siRNA, ShRNA, and miRNA polynucleotides and/or other polynucleotides having RNAi, antisense, ribozyme, or inhibitory functionalities). In addition, anti-viral agents can also include antibodies and binding fragments thereof, and peptides and polypeptides, including peptidomimetics and peptide analogs that modulate and/or target viral gene/protein activity or function in a sequence-specific manner.

Synthesis of anti-viral polynucleotides such as siRNA, shRNA, miRNA, and ribozyme polynucleotides, as well as polynucleotides having modified and mixed backbones, is well-known to those of skill in the art. See e.g. Stein C. A. and Krieg A. M. (eds), Applied SiRNA Oligonucleotide Technology, 1998 (Wiley-Liss). Methods of synthesizing sequence specific antibodies and binding fragments, as well as peptides and polypeptides, including peptidomimetics and peptide analogs, are known to those of skill in the art. See e.g. Lihu Yang et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

In one aspect, the silencing or the downregulation of viral protein expression may be based generally upon the RNAi approach using RNAi polynucleotides (such as siRNA, miRNA, shRNA polynucleotides). These polynucleotides target the viral gene(s)/protein (s) to be silenced and/or downregulated. In certain embodiments, modulation of the viral protein expression comprises the silencing and/or downregulation of the target viral gene and may be based generally upon the siRNA approach using siRNA polynucleotides.

In certain embodiments, the RNAi polynucleotides can inhibit transcription and/or translation of a viral protein. Preferably the polynucleotide is a specific inhibitor of transcription and/or translation from the viral gene, and does not inhibit transcription and/or translation from other genes or mRNAs. The product may bind to the viral gene either (i) 5′ to the coding sequence, and/or (ii) to the coding sequence, and/or (iii) 3′ to the coding sequence.

In certain embodiments, the RNAi polynucleotide, such as siRNA polynucleotide, is directed to a viral protein mRNA. Such a polynucleotide may be capable of hybridizing to the viral mRNA and may thus inhibit the expression of viral by interfering with one or more aspects of viral mRNA metabolism including transcription, mRNA processing, mRNA transport from the nucleus, translation or mRNA degradation. The siRNA polynucleotide typically hybridizes to the viral mRNA to form a duplex which can cause direct inhibition of translation and/or destabilization of the mRNA.

In certain embodiments, the RNAi polynucleotide, such as siRNA polynucleotide, may hybridize to all or part of the target viral RNA. Typically the siRNA polynucleotide hybridizes to the ribosome binding region or the coding region of the viral mRNA. The polynucleotide may be complementary to all of or a region of the viral mRNA. For example, the polynucleotide may be the exact complement of all or a part of viral mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the viral mRNA under conditions of medium to high stringency such as 0.03M sodium chloride and 0.03M sodium citrate at from about 50° C. to about 60° C.

In certain embodiments, suitable polynucleotides are typically from about 19 to 30 nucleotides in length. In other embodiments, a polynucleotide may be from about 19 to about 27 nucleotides in length, or alternatively from about 19 to about 25 nucleotides in length or from about 19 to about 22 nucleotides in length.

The viral protein or proteins targeted by the polynucleotide will be dependent upon the site at which silencing/downregulation is to be effected.

It is also contemplated that polynucleotides targeted to separate viral proteins be used in combination (for example 1, 2, 3, 4 or more different viral proteins may be targeted).

Alternatively, the polynucleotides may be part of compositions that may comprise polynucleotides to more than one viral protein.

Individual siRNA polynucleotides may be specific to a particular viral gene, or may target 1, 2, 3 or more different virals genes according to varying degrees of sequence homology and conserved sequences.

In general, short interfering RNAs (siRNAs) typically comprise 19-27 nucleotide complementary double stranded RNA molecules with 2 nucleotide overhangs on the 3-prime ends of the molecules (de Fougerolles, A., H.-P. Vornlocher, J. Maraganore, and J. Lieberman. 2007. Interfering with disease: a progress report on siRNA-based therapeutics. Nat Rev Drug Discov 6:443-453; Amarzguioui, M., J. J. Rossi, and D. Kim. 2005. Approaches for chemically synthesized siRNA and vector-mediated RNAi. FEBS Letters 579:5974-5981). These compositions can be produced using a variety of tools including, but not limited to chemical synthesis (de Fougerolles, A., H.-P. Vornlocher, J. Maraganore, and J. Lieberman. 2007. Interfering with disease: a progress report on siRNA-based therapeutics. Nat Rev Drug Discov 6:443-453; Ronald, M. 2002. Small Interfering RNAs and Their Chemical Synthesis. Angewandte Chemie International Edition 41:2265-2269; Manoharan, M. 2004. RNA interference and chemically modified small interfering RNAs. Current Opinion in Chemical Biology 8:570-579; Davis, R. H. 1995. Large-scale oligoribonucleotide production. Current Opinion in Biotechnology 6:213-217), shRNA and miRNA expression vectors followed by processing in vivo (Amarzguioui, M., J. J. Rossi, and D. Kim. 2005. Approaches for chemically synthesized siRNA and vector-mediated RNAi. FEBS Letters 579:5974-5981), and in vitro transcription (Sohail, M., G. Doran, J. Riedemann, V. Macaulay, and E. M. Southern. 2003. A simple and cost-effective method for producing small interfering RNAs with high efficacy. Nucl. Acids Res. 31:e38-).

The polynucleotides for use in the invention may suitably be unmodified phosphodiester oligomers. Such polynucleotides may vary in length.

In certain embodiments, the exemplary RNAi polynucleotides may also be chemically modified to improve stability, delivery, and efficacy (Li, C. X., A. Parker, E. Menocal, S. Xiang, L. Borodyansky, and J. H. Fruehauf. 2006. Delivery of RNA interference. Cell Cycle 5:2103-2109). Methods of preparing modified backbone and mixed backbone oligonucleotides are known in the art. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. In certain embodiments, the chemical modifications may include but are not limited to 2′-Oallyl, and 2′-deoxyfluorouridine modifications, phosphothioates, 2′ deoxyfluoridine (2′-F) modification, and locked nucleic acid residues (Li, C. X., A. Parker, E. Menocal, S. Xiang, L. Borodyansky, and J. H. Fruehauf. 2006. Delivery of RNA interference. Cell Cycle 5:2103-2109; de Fougerolles, A., H.-P. Vornlocher, J. Maraganore, and J. Lieberman. 2007. Interfering with disease: a progress report on siRNA-based therapeutics. Nat Rev Drug Discov 6:443-453; Manoharan, M. 2004. RNA interference and chemically modified small interfering RNAs. Current Opinion in Chemical Biology 8:570-579). In certain embodiments, silyl ether protection of the polynucleotide can also be used.

The precise sequence of the siRNA polynucleotide used in the invention will depend upon the target viral protein. In one embodiment, suitable viral siRNA polynucleotides can include polynucleotides such as oligodeoxynucleotides selected from the following sequences set forth in Table 1.

Anti-viral polynucleotides directed to viral proteins can be selected in terms of their nucleotide sequence by any convenient, and conventional, approach. For example, the BLAST search engine at National Center for Biotechnology Information (NCBI). Once selected, the RNAi polynucleotides can be synthesized using a DNA synthesizer.

In one embodiment of the invention, interfering RNA (e.g., siRNA) has a sense strand and an antisense strand, and the sense and antisense strands comprise a region of at least near-perfect contiguous complementarity of at least 19 nucleotides.

In a further embodiment, interfering RNA (e.g., siRNA) has a sense strand and an antisense strand, and the antisense strand comprises a region of at least near-perfect contiguous complementarity of at least 19 nucleotides to a target sequence, and the sense strand comprises a region of at least near-perfect contiguous identity of at least 19 nucleotides with a target sequence of target mRNA, respectively.

The length of each strand of the interfering RNA can comprise 19 to 27 nucleotides, and may comprise a length of 19, 20, 21, 22, 23, 24, 25, 26, or 27 nucleotides.

Interfering RNA target sequences (e.g., siRNA target sequences) within a target mRNA sequence can be selected using available design tools. Interfering RNAs corresponding to a target sequence can then be tested by transfection of cells expressing the target mRNA followed by assessment of knockdown using methods well known in the art.

As used herein, the strands of a double-stranded interfering RNA (e.g., an siRNA) may be connected to form a hairpin or stem-loop structure (e.g., an shRNA).

Nucleotides at the 3′ end of the sense strand may be deoxynucleotides for enhanced processing. Design of dicer-substrate 27-mer duplexes from 19-21 nucleotide target sequences, such as provided herein, is further discussed by the Integrated DNA Technologies (IDT) website and by Kim, D.-H. et al., (February, 2005) Nature Biotechnology 23:2; 222-226.

In certain embodiments, when interfering RNAs are produced by chemical synthesis, phosphorylation at the 5′ position of the nucleotide at the 5′ end of one or both strands (when present) can be added to enhance siRNA efficacy and specificity of the bound RISC complex.

One of skill in the art is able to use the target sequence information provided in Tables 1 or 2 to design interfering RNAs having a length shorter or longer than the sequences provided in the tables.

The target sequence in the mRNAs corresponding to target viral genes may be in the 5′ or 3′ untranslated regions of the mRNA as well as in the coding region of the mRNA.

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single molecule where the regions of complementarity are base-paired and are covalently linked by a hairpin loop so as to form a single strand. It is believed that the hairpin is cleaved intracellularly by a protein termed dicer to form an interfering RNA of two individual base-paired RNA molecules.

Interfering RNAs may differ from naturally-occurring RNA by the addition, deletion, substitution or modification of one or more nucleotides. Non-nucleotide material may be bound to the interfering RNA, either at the 5′ end, the 3′ end, or internally. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Further modifications include a 3′ terminal biotin molecule, a peptide known to have cell-penetrating properties, a nanoparticle, a peptidomimetic, a fluorescent dye, or a dendrimer, for example.

Nucleotides may be modified on their base portion, on their sugar portion, or on the phosphate portion of the molecule and function in embodiments of the present invention.

Modifications include substitutions with alkyl, alkoxy, amino, deaza, halo, hydroxyl, thiol groups, or a combination thereof, for example. Nucleotides may be substituted with analogs with greater stability such as replacing a ribonucleotide with a deoxyribonucleotide, or having sugar modifications such as 2′ OH groups replaced by 2′ amino groups, 2′ O-methyl groups, 2′ methoxyethyl groups, or a 2′-O, 4′-C methylene bridge, for example. Examples of a purine or pyrimidine analog of nucleotides include a xanthine, a hypoxanthine, an azapurine, a methylthioadenine, 7-deaza-adenosine and O- and N-modified nucleotides. The phosphate group of the nucleotide may be modified by substituting one or more of the oxygens of the phosphate group with nitrogen or with sulfur (phosphorothioates). Modifications are useful, for example, to enhance function, to improve stability or permeability, or to direct localization or targeting.

There may be a region or regions of the antisense interfering RNA strand that is (are) not complementary to a portion of the target viral genes. Non-complementary regions may be at the 3′, 5′ or both ends of a complementary region or between two complementary regions.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers such as Ambion Inc. (Austin, Tex.), Invitrogen (Carlsbad, Calif.), or Dharmacon (Lafayette, Colo.). Interfering RNAs are purified by extraction with a solvent or resin, precipitation, electrophoresis, chromatography, or a combination thereof, for example. Alternatively, interfering RNA may be used with little if any purification to avoid losses due to sample processing.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT®DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Minis, Madison, Wis.). A first interfering RNA may be administered via in vivo expression from a first expression vector capable of expressing the first interfering RNA and a second interfering RNA may be administered via in vivo expression from a second expression vector capable of expressing the second interfering RNA, or both interfering RNAs may be administered via in vivo expression from a single expression vector capable of expressing both interfering RNAs.

Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

Hybridization under Physiological Conditions: In certain embodiments of the present invention, an antisense strand of an interfering RNA hybridizes with an mRNA in vivo as part of the RISC complex.

For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning: A Laboratory Manual, Cold Spring Harbor Laboratory Press, Cold Spring Harbor, N.Y. Further examples of stringent hybridization conditions include 400 mM NaCl, 40 mM PIPES pH 6.4, 1 mM EDTA, 50° C. or 70° C. for 12-16 hours followed by washing, or hybridization at 70° C. in 1×SSC or 50° C. in 1×SSC, 50% formamide followed by washing at 70° C. in 0.3×SSC, or hybridization at 70° C. in 4×SSC or 50° C. in 4×SSC, 50% formamide followed by washing at 67° C. in 1×SSC. The temperature for hybridization is about 5-10° C. less than the melting temperature (Tₘ) of the hybrid where Tₘ is determined for hybrids between 19 and 49 base pairs in length using the following calculation: Tₘ° C.=81.5+16.6(log₁₀[Na+])+0.41 (% G+C)−(600/N) where N is the number of bases in the hybrid, and [Na+] is the concentration of sodium ions in the hybridization buffer.

Single-stranded interfering RNA: As cited above, interfering RNAs ultimately function as single strands. Single-stranded (ss) interfering RNA has been found to effect mRNA silencing, albeit less efficiently than double-stranded RNA. Therefore, embodiments of the present invention also provide for administration of a ss interfering RNA that hybridizes under physiological conditions to a portion of the target RNA.

SS interfering RNAs are synthesized chemically or by in vitro transcription or expressed endogenously from vectors or expression cassettes as for ds interfering RNAs. 5′ Phosphate groups may be added via a kinase, or a 5′ phosphate may be the result of nuclease cleavage of an RNA. Delivery is as for ds interfering RNAs. In one embodiment, ss interfering RNAs having protected ends and nuclease resistant modifications are administered for silencing. SS interfering RNAs may be dried for storage or dissolved in an aqueous solution. The solution may contain buffers or salts to inhibit annealing or for stabilization.

Hairpin interfering RNA: A hairpin interfering RNA is a single molecule (e.g., a single oligonucleotide chain) that comprises both the sense and antisense strands of an interfering RNA in a stem-loop or hairpin structure (e.g., an shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Techniques for selecting target sequences for siRNAs are provided by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNAs.

Polynucleotide Homologues

Homology and homologues are discussed herein (for example, the polynucleotide may be a homologue of a complement to a sequence in viral mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 25 contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by no more than about 1, 2, 3, 4, 5, 6, 7, 8, 9, 10, 15, or 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Viral Agents

As used herein, polypeptide and polypeptide anti-viral agents can include binding proteins, including peptides, peptidomimetics, antibodies, antibody fragments, and the like, are also suitable modulators of viral gene/protein functions. Exemplary peptide and polypeptide anti-viral agents can modulate the structure, function and/or activity of the viral proteins encoded by the sequences and sequence homologs thereof as described in Table 2.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. viral infection and/or replication protein or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

General Methodology

Aspects and embodiments of the present disclosure is directed to methods and compositions that contain double stranded RNA (“dsRNA”), and methods of use thereof that are capable of reducing the expression of target genes in eukaryotic cells. One of the strands of the dsRNA contains a region of nucleotide sequence that has a length that ranges from about 19 to about 27 nucleotides that can direct the destruction of the RNA transcribed from the target gene.

Using DNA sequence information from a variety of different poxvirus genomes, candidate siRNA molecules specific for many different poxvirus genes were designed and synthesized. The resulting siRNAs were tested for their abilities to inhibit the replication of both Vaccinia virus and Cowpoxvirus in vitro. siRNA molecules specific for at least 7 different viral genes were able to efficiently block poxvirus replication in vitro. These studies revealed that: 1) RNAi can be used to inhibit the replication of poxviruses, and 2) transfection of cells with siRNAs can dramatically reduce the ability of both Vaccinia virus and Cowpoxvirus to replicate. These data demonstrate that poxvirus-specific siRNAs can be useful for the treatment of animal and human infection, and can be used to identify viral genes that are targets for other potentially useful therapies such as small molecule inhibitors.

Utilizing an in vitro virus replication reduction assay, we have identified at least 13 siRNAs that reduce expression of at least 7 vaccinia virus genes and subsequently inhibit vaccinia virus replication. These specific siRNAs and other siRNAs that target the poxvirus genes we identified can be used to inhibit vaccinia and other poxvirus replication and protect against poxvirus infection in vivo.

The instant disclosure is directed to a number of unique compositions for poxvirus prophylactic and therapeutic compounds. First, utilizing an in vitro vaccinia virus replication assay in conjunction with siRNA-mediated gene silencing targeting a large number of poxvirus genes (Table 1), we have identified at least 7 poxvirus genes that are required for poxvirus replication (Table 2) (Upton et. al. JV 2003). All of these genes are novel anti-viral drug targets for poxvirus and related viruses. Second, we have identified specific siRNA sequences that can block expression of poxvirus genes and inhibit poxvirus replication. These siRNAs individually or combined, represent target sequences that could be used for design and synthesis of chemically modified or unmodified siRNAs, micro RNAs or other nucleic acid-based gene regulation.

TABLE 1
Poxvirus genes targeted by siRNA molecules

| | | Genomic | siRNAs |
|---|---|---|---|
| Gene | Locus Tag | sequence* | tested† |
| A7L | VACWR126 | 115797-117929 | n = 3 |
| A9L | VACWR128 | 118842-119168 | n = 3 |
| A17L | VACWR137 | 125583-126194 | n = 2 |
| A22R | VACWR142 | 129467-130030 | n = 3 |
| A26L | VACWR146 | 134860-135324 | n = 2 |
| A28L | VACWR151 | 140346-140786 | n = 2 |
| A29L | VACWR152 | 140787-141704 | n = 3 |
| A32L | VACWR155 | 142401-143213 | n = 2 |
| A41L | VACWR166 | 149505-150164 | n = 2 |
| A56R | VACWR181 | 162183-163127 | n = 2 |
| B8R | VACWR190 | 170571-171389 | n = 2 |
| B13R | VACWR195 | 173473-174510 | n = 2 |
| B14R | VACWR195 | 172887-173555 | n = 2 |
| C3L | VACWR025 | 18677-19468 | n = 2 |
| D5R | VACWR110 | 98275-100632 | n = 2 |
| D6R | VACWR111 | 100673-102586 | n = 3 |
| D7R | VACWR112 | 102613-103098 | n = 3 |
| E1L | VACWR057 | 44004-45443 | n = 1 |
| E9L | VACWR065 | 53636-56656 | n = 5 |
| F10L | VACWR049 | 36459-37778 | n = 3 |
| G1L | VACWR078 | 68977-70752 | n = 2 |
| G4L | VACWR081 | 71710-72084 | n = 1 |
| I4L | VACWR073 | 61925-64240 | n = 2 |
| I7L | VACWR076 | 65666-66937 | n = 5 |
| J5L | VACWR097 | 82857-83258 | n = 3 |
| K1L | VACWR032 | 25071-25925 | n = 2 |
| L4R | VACWR091 | 79139-79894 | n = 3 |
| L5R | VACWR092 | 79904-80290 | n = 3 |
| N1L | VACWR028 | 21819-22172 | n = 2 |

Table 1 lists siRNAs that inhibited vaccinia virus replication in vitro. The percent inhibition of virus replication, gene target, and siRNA sequence are shown in Table 2.

TABLE 2
Viral gene-specific siRNA molecules that inhibit Orthopoxvirus
replication

| | | | | | |
|---|---|---|---|---|---|
| | | % inhibition | | SEQ | |
| | | (each | | ID | Gene |
| siRNA | Replicates | experiment) | siRNA Sequence | NOS | accession # |

| | | | | | |
|---|---|---|---|---|---|
| F10L | 2 | 34.8 | 5′UGGGCUCCGUCAGUUAGAUUGUUAA | 1 | AY243312 |
| #1 | | 78.9 | 5′UUAACAAUCUAACUGACGGAGCCCA | 2 | (36459- |
| | | | | | 37778) |
| F10L | 2 | 35.2 | 5′CACAUAUCUACAGGAGGAUAUGGUA | 3 | AY243312 |
| #2 | | 50.5 | 5′UACCAUAUCCUCCUGUAGAUAUGUG | 4 | (36459- |
| | | | | | 37778) |
| A17L | 2 | 66.8 | 5′CCUAAAGAUGGAGGUAUGAUGCAAA | 5 | AY243312 |
| #1 | | 74 | 5′UUUGCAUCAUACCUCCAUCUUUAGG | 6 | (125583- |
| | | | | | 126194) |
| A17L | 3 | 76.4 | 5′UGGCUCUAUAUAGCCCUCCUCUAAU | 7 | AY243312 |
| #2 | | 65.3 | 5′AUUAGAGGAGGGCUAUAUAGAGCCA | 8 | (125583- |
| | | 86.8 | | | 126194) |
| A29L | 3 | 47.5 | 5′CGACCGAGUUAAAGGAAACUUUGUU | 9 | AY243312 |
| #3 | | 48.4 | 5′AACAAAGUUUCCUUUAACUCGGUCG | 10 | (140787- |
| | | 89.6 | | | 141704) |
| G1L | 2 | 38.6 | 5′GCAACGGAAUCGGACGCAAUCAGAA | 11 | AY243312 |
| #1 | | 68.5 | 5′UUCUGAUUGCGUCCGAUUCCGUUGC | 12 | (68977- |
| | | | | | 70752) |
| E1L | 2 | 49.7 | 5′CGGACAUAUUAGGAGUUCUUACUAU | 13 | AY243312 |
| #2 | | 60.2 | 5′AUAGUAAGAACUCCUAAUAUGUCCG | 14 | (44004- |
| | | | | | 45443) |
| E1L | 2 | 39.2 | 5′UAGUGGAUCCGACGUUUCAACUAUU | 15 | AY243312 |
| #3 | | 46.3 | 5′AAUAGUUGAAACGUCGGAUCCACUA | 16 | (44004- |
| | | | | | 45443) |
| D7R | 2 | 37 | 5′CCUCAUGAGCUGACGUUAGACAUAA | 17 | AY243312 |
| #2 | | 61 | 5′UUAUGUCUAACGUCAGCUCAUGAGG | 18 | (102613- |
| | | | | | 103098) |
| D5R | 3 | 35.3 | 5′CGGCUAUUAGAGGUAAUGAUGUUAU | 19 | AY243312 |
| #1 | | 52.1 | 5′AUAACAUCAUUACCUCUAAUAGCCG | 20 | (98275- |
| | | 99 | | | 100632) |
| D5R | 2 | 40.1 | 5′UAGGGAUGAGGAAGCAUACUCUAUA | 21 | AY243312 |
| #2 | | 63.3 | 5′UAUAGAGUAUGCUUCCUCAUCCCUA | 22 | (98275- |
| | | 99 | | | 100632) |
| A9L | 2 | 36.6 | 5′CAAGUAGCCAAUGGCGCCAUAGAUU | 23 | AY243312 |
| #1 | | 62.4 | 5′AAUCUAUGGCGCCAUUGGCUACUUG | 24 | (118842- |
| | | | | | 119168) |
| A9L | 2 | 67.7 | 5′GAGCCAUUGCGAGCAUGAUAAUGUA | 25 | AY243312 |
| #2 | | 77.3 | 5′UACAUUAUCAUGCUCGCAAUGGCUC | 26 | (118842- |
| | | | | | 119168) |
| A56R | 2 | 53.8 | 5′CAUCGCCUACAAAUGACACUGAUAA | 27 | AY243312 |
| #1 | | 97.2 | 5′UUAUCAGUGUCAUUUGUAGGCGAUG | 28 | (162183- |
| | | | | | 163127) |
| A29L | 2 | 30.3 | 5′ACUCGAACCCUCAUUGGCUACAUUU | 29 | AY243312 |
| #1 | | 33.8 | 5′AAAUGUAGCCAAUGAGGGUUCGAGU | 30 | (140787- |
| | | | | | 141704) |
| L4R | 2 | 63.4 | 5′CCUCAUCGAAGAAGAUACCAUAUUU | 31 | AY243312 |
| #1 | | 87 | 5′AAAUAUGGUAUCUUCUUCGAUGAGG | 32 | (79139- |
| | | | | | 79894) |
| G4L | 2 | 72.5 | 5′CCGAGUAUGAUAUACUCCAUGUUGA | 33 | AY243312 |
| | | 95.4 | 5′UCAACAUGGAGUAUAUCAUACUCGG | 34 | (71710- |
| | | | | | 72084) |
| E9L | 2 | 46.6 | 5′CCACAGGUAAUUAUGUGACUGUUGA | 35 | AY243312 |
| #4 | | 39.2 | 5′UCAACAGUCACAUAAUUACCUGUGG | 36 | (53636- |
| | | | | | 56656) |

Targeted inhibition of gene expression may be implemented via the use of RNA interference molecules, where the nucleotide sequence of such compounds are related to the nucleotide sequences of DNA and/or RNA of genes that are involved in the initiation, transcription, translation or replication of poxviruses. In preferred embodiments, an RNA interference (RNAi) molecule is used to decrease gene expression in a poxvirus. The methods described herein are generally applicable to any of the Poxyiridae. The methods of the invention are preferably used to silence gene expression and replication in the Orthopoxvirus genera, including, but not limited to, camelpox, cowpox, monkeypox, vaccinia, and variola.

Dosage/Formulation/Administration

Pharmaceutical compositions for use in accordance with the present invention may be formulated in conventional manner using one or more physiologically acceptable carriers or excipients. Thus, the RNA molecules of the present invention may be formulated for administration by inhalation or insufflation (either through the mouth or the nose) (Thomas, M., J. J. Lu, J. Chen, and A. M. Klibanov. 2007. Non-viral siRNA delivery to the lung. Advanced Drug Delivery Reviews 59:124-133) or oral, parenteral or mucosal (such as buccal, vaginal, rectal, sublingual) or intravenous (Herweijer, H., and J. A. Wolff 2006. Gene therapy progress and prospects: Hydrodynamic gene delivery. Gene Ther 14:99-107; Li, S. D., and L. Huang. Gene therapy progress and prospects: non-viral gene therapy by systemic delivery. Gene Ther 13:1313-1319) administration (Li, C. X., A. Parker, E. Menocal, S. Xiang, L. Borodyansky, and J. H. Fruehauf 2006. Delivery of RNA interference. Cell Cycle 5:2103-2109; de Fougerolles, A., H.-P. Vornlocher, J. Maraganore, and J. Lieberman. 2007. Interfering with disease: a progress report on siRNA-based therapeutics. Nat Rev Drug Discov 6:443-453). Methods of preparing pharmaceutical formulations are well known (Li, C. X., A. Parker, E. Menocal, S. Xiang, L. Borodyansky, and J. H. Fruehauf 2006. Delivery of RNA interference. Cell Cycle 5:2103-2109). Dosage of inhibitory nucleic acids may vary by route of administration, exemplary suitable dosages can range from about 0.1 mg/kg to about 3 mg/kg or greater.

The inhibitory polynucleotide molecules may be naked (i.e. non-formulated) or formulated in a variety of carrier agents such as, but not limited to, polyethylenimine (PEI) and other polymers (Thomas, M., J. J. Lu, J. Chen, and A. M. Klibanov. 2007. Non-viral siRNA delivery to the lung. Advanced Drug Delivery Reviews 59:124-133; Howard, K. A., and J. Kjems. 2007. Polycation-based nanoparticle delivery for improved RNA interference therapeutics. Expert Opinion on Biological Therapy 7:1811-1822), nanoparticles, cationic lipids/liposomes (DOTAP, DOPE, cholesterol, etc.) (Howard, K. A., and J. Kjems. 2007. Polycation-based nanoparticle delivery for improved RNA interference therapeutics. Expert Opinion on Biological Therapy 7:1811-1822; Zhang, S., B. Zhao, H. Jiang, B. Wang, and B. Ma. 2007. Cationic lipids and polymers mediated vectors for delivery of siRNA. Journal of Controlled Release 123:1-10), peptide (Meade, B. R., and S. F. Dowdy. 2007. Exogenous siRNA delivery using peptide transduction domains/cell penetrating peptides. Advanced Drug Delivery Reviews 59:134-140; Moschos, S. A., A. E. Williams, and M. A. Lindsay. 2007. Cell-penetrating-peptide-mediated siRNA lung delivery. Biochemical Society Transactions 035:807-810) (e.g. Penetratin™ 1 (MPBiomedicals, Solon, Ohio)) protein/immunoglobulin (Liu, B. 2007. Exploring cell type-specific internalizing antibodies for targeted delivery of siRNA. Brief Funct Genomic Proteomic 6:112-119), or polyelectrolyte transfection reagents. Compositions may include conjugation of carriers (e.g. peptides or cholesterols) or formulation (mixing).

In certain embodiments, the RNAi polynucleotides may also be combined with other therapeutic agents or compounds (e.g. antibiotics) as co-administration or co-formulation components.

In certain embodiments, the pharmaceutical formulations comprise interfering RNAs, or salts thereof, of the invention up to 99% by weight mixed with a physiologically acceptable carrier medium such as water, buffer, saline, glycine, hyaluronic acid, mannitol, and the like.

Interfering RNA embodiments of the present invention can be administered as solutions, suspensions, or emulsions.

Generally, an effective amount of the interfering RNAs of embodiments of the invention results in an extracellular concentration at the surface of the target cell of from 100 μM to 1000 nM, or from 1 nM to 400 nM, or from 5 nM to about 100 nM, or about 10 nM. The dose required to achieve this local concentration will vary depending on a number of factors including the delivery method, the site of delivery, the number of cell layers between the delivery site and the target cell or tissue, whether delivery is local or systemic, etc. The concentration at the delivery site may be considerably higher than it is at the surface of the target cell or tissue.

Topical compositions are delivered to the surface of the target organ one to four times per day, or on an extended delivery schedule such as daily, weekly, bi-weekly, monthly, or longer, according to the routine discretion of a skilled clinician. The pH of the formulation is about pH 4-9, or pH 4.5 to pH 7.4.

An effective amount of a formulation may depend on factors such as the age, race, and sex of the subject, the severity of the virial infection, the rate of target gene transcript/protein turnover, the interfering RNA potency, and the interfering RNA stability, for example. In one embodiment, the interfering RNA is delivered topically to a target organ and reaches target protein-containing tissue at a therapeutic dose thereby ameliorating a viral-infection/replication process.

Acceptable carriers: An acceptable carrier refers to those carriers that cause at most, little to no ocular irritation, provide suitable preservation if needed, and deliver one or more interfering RNAs of the present invention in a homogenous dosage. An acceptable carrier for administration of interfering RNA of embodiments of the present invention include the cationic lipid-based transfection reagents TransIT®-TKO (Minis Corporation, Madison, Wis.), LIPOFECTIN®, Lipofectamine, OLIGOFECTAMINE® (Invitrogen, Carlsbad, Calif.), or DHARMAFECT® (Dharmacon, Lafayette, Colo.); polycations such as polyethyleneimine; cationic peptides such as Tat, polyarginine, or Penetratin (Antp peptide); or liposomes. Liposomes are formed from standard vesicle-forming lipids and a sterol, such as cholesterol, and may include a targeting molecule such as a monoclonal antibody having binding affinity for endothelial cell surface antigens, for example. Further, the liposomes may be PEGylated liposomes.

The exemplary interfering RNAs may be delivered in solution, in suspension, or in bioerodible or non-bioerodible delivery devices. The interfering RNAs can be delivered alone or as components of defined, covalent conjugates. The interfering RNAs can also be complexed with cationic lipids, cationic peptides, or cationic polymers; complexed with proteins, fusion proteins, or protein domains with nucleic acid binding properties (e.g., protamine); or encapsulated in nanoparticles. Tissue- or cell-specific delivery can be accomplished by the inclusion of an appropriate targeting moiety such as an antibody or antibody fragment.

For ophthalmic, otic, or pulmonary delivery, an exemplary interfering RNA may be combined with opthalmologically, optically, or pulmonary acceptable preservatives, co-solvents, surfactants, viscosity enhancers, penetration enhancers, buffers, sodium chloride, or water to form an aqueous, sterile suspension or solution. Solution formulations may be prepared by dissolving the interfering RNA in a physiologically acceptable isotonic aqueous buffer. Further, the solutions may include an acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the present invention to improve the retention of the compound.

In certain embodiments, preparation of a sterile ointment formulation can include the combination of the exemplary interfering RNA with a preservative in an appropriate vehicle, such as mineral oil, liquid lanolin, or white petrolatum.

Sterile gel formulations may be prepared by suspending the interfering RNA in a hydrophilic base prepared from the combination of, for example, CARBOPOL®-940 (BF Goodrich, Charlotte, N.C.), or the like, according to methods known in the art. VISCOAT® (Alcon Laboratories, Inc., Fort Worth, Tex.) may be used for intraocular injection, for example.

Other compositions of the present invention may contain penetration enhancing agents such as cremephor and TWEEN® 80 (polyoxyethylene sorbitan monolaureate, Sigma Aldrich, St. Louis, Mo.), in the event the interfering RNA is less penetrating in the organ or tissue of interest.

An embodiment of the invention also includes an expression vector comprising a polynucleotide encoding siRNA sequence of the invention in a manner that allows expression of the polynucleotide (Li, C. X., A. Parker, E. Menocal, S. Xiang, L. Borodyansky, and J. H. Fruehauf. 2006. Delivery of RNA interference. Cell Cycle 5:2103-2109; de Fougerolles, A., H.-P. Vornlocher, J. Maraganore, and J. Lieberman. 2007. Interfering with disease: a progress report on siRNA-based therapeutics. Nat Rev Drug Discov 6:443-453; Amarzguioui, M., J. J. Rossi, and D. Kim. 2005. Approaches for chemically synthesized siRNA and vector-mediated RNAi. FEBS Letters 579:5974-5981). An embodiment of the invention also includes a host cell, for example a human cell, including an expression vector contemplated by the invention. In yet another embodiment, an expression vector of the invention comprises a nucleic acid sequence encoding two or more siRNA sequences, which can be the same or different.

Routes of Administration

A variety of protocols are available for in vivo delivery and administration of the exemplary RNAi polynucleotides. Inhibitory nucleic acids have been applied successfully in vivo in animal models for a variety of infectious diseases including influenza virus (Tompkins, S. M., C. Y. Lo, T. M. Tumpey, and S. L. Epstein. 2004. Protection against lethal influenza virus challenge by RNA interference in vivo. Proc Natl Acad Sci USA 101:8682-8686) and respiratory synctial virus (Bitko, V., A. Musiyenko, O, Shulyayeva, and S. Batik. 2005. Inhibition of respiratory viruses by nasally administered siRNA. Nat Med 11:50-55). Moreover, inhibitory nucleic acids are in human clinical trial for a variety of diseases, including RSV, HIV, and Acute Macular Degeneration (AMD) (de Fougerolles, A., H.-P. Vornlocher, J. Maraganore, and J. Lieberman. 2007. Interfering with disease: a progress report on siRNA-based therapeutics. Nat Rev Drug Discov 6:443-453; Rossi, J. J., C. H. June, and D. B. Kohn. 2007. Genetic therapies against HIV. Nat Biotech 25:1444-1454).

In certain embodiments, interfering RNA may be delivered, for example, via aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal administration. In certain other embodiments, administration may be directly to the lungs, via, for example, an aerosolized preparation, and by inhalation via an inhaler or a nebulizer, for example.

The compositions can be delivery prophylactically (i.e. prior to exposure or infection to reduce the likelihood or severity of infection) or therapeutically (after infection). Composition treatment can be a stand alone monotherapy or be given in combination with anti-poxvirus therapies (e.g., Vaccinia Immune Globulin Intravenous (VIGIV; DynPort Vaccine Company LLC, Frederick, Md.).

In certain further embodiments, modes of administration can include tablets, pills, and capsules, all of which are capable of formulation by one of ordinary skill in the art.

Kits:

Aspects and embodiments of the present disclosure also provide a kit that includes reagents for attenuating the expression of an mRNA in a subject. The kit can contain an siRNA, miRNA or an shRNA expression vector. For siRNAs and non-viral shRNA expression vectors the kit also may contain a transfection reagent or other suitable delivery vehicle. For viral shRNA expression vectors, the kit may contain the viral vector and/or the necessary components for viral vector production (e.g., a packaging cell line as well as a vector comprising the viral vector template and additional helper vectors for packaging). The kit may also contain positive and negative control siRNAs or shRNA expression vectors (e.g., a non-targeting control siRNA or an siRNA that targets an unrelated mRNA). The kit also may contain reagents for assessing knockdown of the intended target gene (e.g., primers and probes for quantitative PCR to detect the target mRNA and/or antibodies against the corresponding protein for western blots). Alternatively, the kit may comprise an siRNA sequence or an shRNA sequence and the instructions and materials necessary to generate the siRNA by in vitro transcription or to construct an shRNA expression vector.

In certain embodiments, the instant disclosure provides a pharmaceutical pack or kit comprising one or more containers filled with the RNA molecules of the present invention. Optionally associated with such container(s) can be a notice in the form prescribed by a governmental agency regulating the manufacture, use or sale of pharmaceuticals or biological products, which notice reflects approval by the agency of manufacture, use or sale for human administration. An embodiment of the invention provides kits that can be used in the above methods. In one embodiment, a kit comprises RNA molecules of the present invention, in one or more containers, and one or more other prophylactic or therapeutic agents useful for the treatment of poxvirus, in one or more containers.

In certain other embodiments, a pharmaceutical combination for co-administration or co-formulation is provided that includes, for example, a packaged combination comprising: an interfering RNA composition, a therapeutic agent, and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### Example 1

Vero E6 cells (CRL-1586) purchased from ATCC (Manassas, Va.) were plated in standard, tissue culture treated, 12-well plates. Once the cells were ±90% confluent, the cells were transfected with control or experimental siRNAs (100 nM final concentration) using Lipofectamine™ 2000 transfection reagent (Invitrogen), following the manufacturers instructions. 16-24 hours later the cells were infected with a recombinant vaccinia virus that expresses GFP (Vac-GFP). In brief, cells were washed with phosphate-buffered saline (PBS) and infected at an MOI of ±0.2 PFU of Vac-GFP/cell in 100 μl PBS. Cells were infected for 1 hour and then 1 ml of culture medium was added to the wells and the plates were incubated in a 37° C., 5.0% CO₂ incubator. After 18-24 hours, fluorescence was visualized using a Typhoon™ 9400 Multi-Format Imager (Amersham Biosciences). Fluorescence directly correlates with Vac-GFP replication. Vero E6 cells treated with Lipofectamine™ 2000 without siRNA and uninfected cells were used to determine background fluorescence. Vero E6 cells transfected with a fluNP-specific siRNA using Lipofectamine™ 2000 in infected cells with Vac-GFP were the negative control and used to determine maximum Vac-GFP replication. Vero E6 cells transfected with a GFP-specific siRNA using Lipofectamine™ 2000 in infected cells with Vac-GFP were the positive control. While the GFP-specific siRNA did not inhibit vaccinia virus replication, it did inhibit GFP expression and was used to determine minimum GFP-expression and fluorescence. Percent inhibition was calculated with the following formula:

$$Percent inhibition = 100 \% x \frac{( Experimental siRNA fluorescence - background fluorescence )}{( fluNP siRNA fluorescence - background fluorescence )}$$

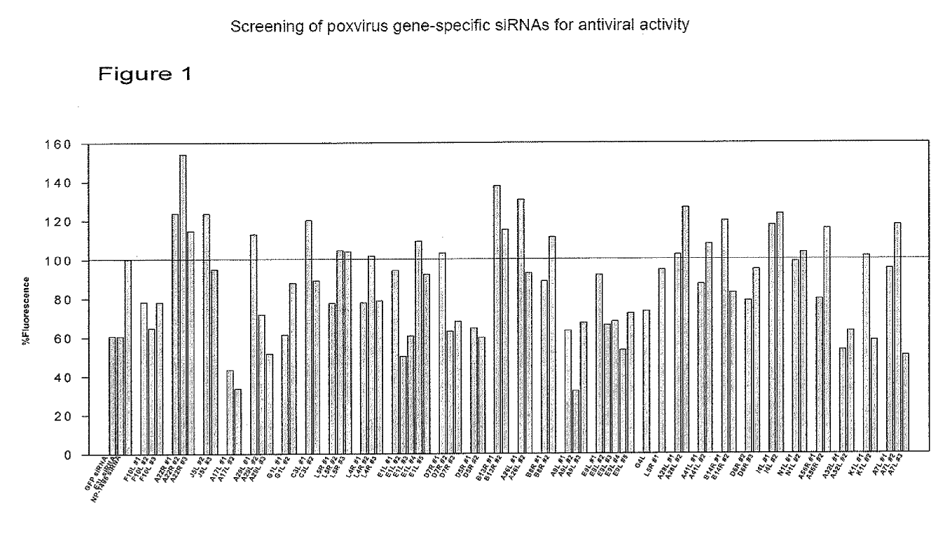

Greater than 80 individual siRNAs were screened using this assay. Percent inhibition for representative siRNAs are shown in FIG. 1.

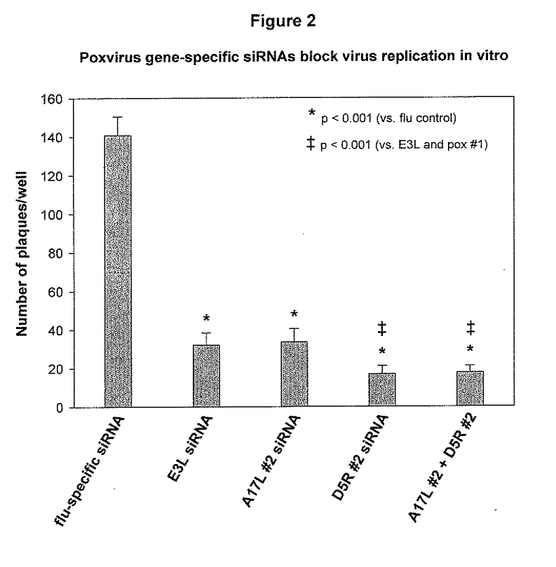

From this primary screen, 13 siRNAs were selected that inhibited Vac-GFP replication (Table 2). Inhibition of Vac-GFP replication by these siRNAs was confirmed in repeat experiments (Table 2) and then tested in a classic plaque assay (FIG. 2). In these assays, an additional, control siRNA was included; a well-characterized influenza nucleoprotein-specific siRNA, NP-1496 (Ge et al., Proc Natl Acad Sci USA. 2003; 100(5):2718-23; Ge et al., Proc Natl Acad Sci USA. 2004; 101(23):8676-81; Tompkins et al., Proc Natl Acad Sci USA. 2004; 101(23):8682-6).

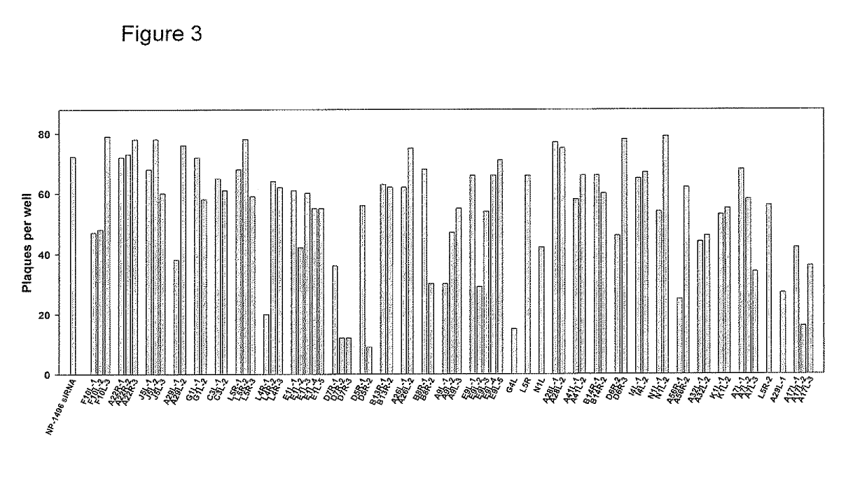

All of the described siRNAs were tested in a wildtype vaccinia virus model of infection. In brief, Vero E6 cells were plated and transfected as above. 16-24 hours later the cells were infected with poxvirus. 48-72 hours later, the cells were fixed with methanol, and the plaques visualized by negative staining with crystal violet (FIG. 3).

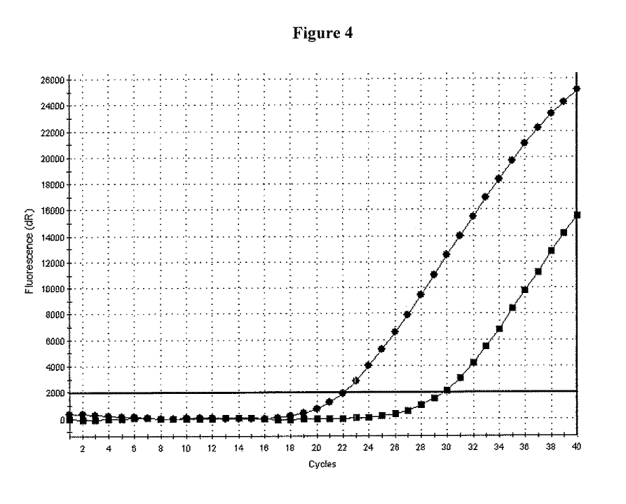

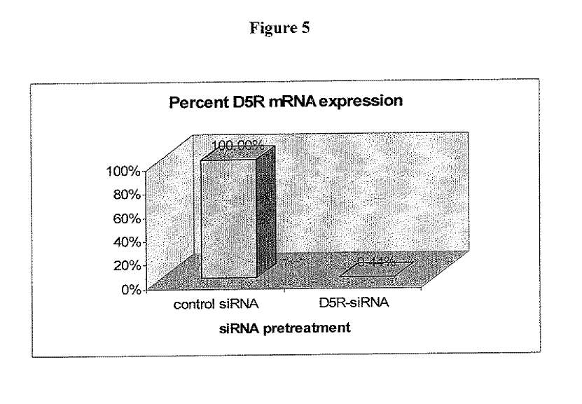

Vero E6 cells are known to have a complete deletion in the interferon locus (Diaz et al., Proc Natl Acad Sci USA. 1988; 85(14):5259-63; Diaz et al., Genomics. 1994; 22(3):540-52), so any reduction in viral replication cannot be the result of a non-specific anti-viral interferon response induced by the transfection with siRNA (Ge et al., Proc Natl Acad Sci USA. 2003; 100(5):2718-23). As multiple siRNAs targeting each gene successfully reduced poxvirus replication, it is unlikely the observed reduction was due to a non-specific or “off-target” effect. However, to confirm that the siRNAs were specifically silencing the target viral mRNA, RNA was purified from infected cells and assayed by real-time, reverse-transcription polymerase chain reaction (RT-PCR) for viral mRNA levels. A sample amplification plot and bar graph showing >227-fold reduction in pox gene D5R mRNA levels by D5R-specific siRNA pre-treatment are shown in FIG. 4 and FIG. 5, respectively.

### Example 2

### Inhaled or Topical Pulmonary Delivery of Anti-Viral Compositions

The animal is lightly anesthetized and suitable exemplary inhibitory nucleic acid is delivered by droplet in the nose (e.g. in a 0.05 ml volume for a mouse) at a concentration of, for example, 2.5 mg/kg (0.05 mg for a 20 g mouse) formulated in phosphate buffered saline (PBS). The composition is inhaled by the anesthetized animal. Twenty four hours after prophylactic treatment, the animal is challenged by respiratory infection with a suitable dose of live poxvirus and the animal is monitored for disease. Experimental protocol can vary by dose, addition of delivery formulations (e.g. PEI, liposomes, etc.), or the time between treatment and infection accordingly.

### Example 3

### Systemic Delivery of Compositions

Exemplary methods for systemic delivery of compositions described herein can be targeted to multiple tissues, including lung, liver, spleen, kidney. Suitable exemplary inhibitory nucleic acid (formulated or non-formulated) is delivered in saline via intravenous delivery utilizing hydrodynamic injection (Herweijer, H., and J. A. Wolff. 2006. Gene therapy progress and prospects: Hydrodynamic gene delivery. Gene Ther 14:99-107; Lewis, D. L., and J. A. Wolff. 2007. Systemic siRNA delivery via hydrodynamic intravascular injection. Adv Drug Deliv Rev 59:115-123). The animal is challenged by respiratory infection with a suitable dose of live poxvirus and the animal is monitored for disease.

Each of these protocols is possible, but other in vivo delivery protocols, while not described, are also available. In all cases, endpoints for disease may include tissue virus titer, tissue pathology, animal weight loss, and animal survival. Composition treatment would reduce tissue virus titer, tissue pathology, and animal weight loss, while improving animal survival.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

While a particular embodiment of the invention has been shown and described, numerous variations and alternate embodiments will occur to those skilled in the art. Accordingly, it is intended that the invention be limited only in terms of the appended claims.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope.

Other embodiments are within the following claims. In addition, where features or aspects of the invention are described in terms of Markush groups, those skilled in the art will recognize that the invention is also thereby described in terms of any individual member or subgroup of members of the Markush group.

Vaccinia Virus F10L gene sequence
Genbank-AY243312
Bases 36459-37778
SEQ ID NO 37:
ttagtttccgccatttatccagtctgagaaaatgtctctcataataaatt
tttccaagaaactaattgggtgaagaatggaaacctttaatctatattta
tcacagtctgttttggtacacatgatgaattcttctaatgctgtactaaa
ttcgatatctttttcgatttctggatatgtttttaataaagtatgaacaa
agaaatggaaatcgtaataccagttatgttcaactttgaaattgtttttt
attttcttgttaatgattccagccacttgggaaaagtcaaagtcgtttaa
tgccgatttaatacgttcattaaaaacaaactttttatcctttagatgaa
ttattattggttcattggaatcaaaaagtaagatattatcgggtttaaga
tctgcgtgtaaaaagttgtcgcaacagggtagttcgtagattttaatgta
taacagagccatctgtaaaaagataaactttatgtattgtaccaaagatt
taaatcctaatttgatagctaactcggtatctactttatctgccgaatac
agtgctaggggaaaaattataatatttcctctttcgtattcgtagttagt
tctcttttcatgttcgaaaaagtgaaacatgcggttaaaatagtttataa
cattaatattactgttaataactgccggataaaagtgggatagtaatttc
acgaatttgatactgtcctttctctcgttaaacgcctttaaaaaaacttt
agaagaatatctcaatgagagttcctgaccatccatagtttgtatcaata
atagcaacatatgaagaacccgtttatacagagtatgtaaaaatgttaat
ttatagtttaatcccatggcccacgcacacacgattaattttttttcatc
tccctttagattgttgtatagaaatttgggtactgtgaactccgccgtag
tttccatgggactatataattttgtggcctcgaatacaaattttactaca
tagttatctatcttaaagactataccatatcctcctgtagatatgtgata
aaaatcgtcgtttataggataaaatcgtttatccttttgttggaaaaagg
atgaattaatgtaatcattctcttctatctttagtagtgtttccttatta
aaattcttaaaataatttaacaatctaactgacggagcccaattttggtg
taaatctaattgggacattatgttgttaaaatacaaacagtctcctaata
taacagtatctgataatctatggggagacatccattgatattcaggggat
gaatcattggcaacacccat
Vaccinia Virus A17L gene sequence
Genbank-AY243312
Bases 125583-126194
SEQ ID NO 38:
atgagttatttaagatattacaatatgcttgacgacttctctgcgggtgc
tggagtgcttgataaagatttatttacagaggaacagcagcaatcgttta
tgcctaaagatggaggtatgatgcaaaacgattatggaggaatgaatgat
tatttgggaatcttcaaaaataatgatgttagaacgttactcggtttgat
tttgttcgtcttggctctatatagccctcctctaatctctatattgatga
tatttatctcatcttttctattgcctcttactagcttagttattacctat
tgcttagtaactcaaatgtatcgtggaggtaatggcaacactgtgggaat
gtctattgtgtgtattgtagctgctgtaattattatggcaatcaatgtat
ttacgaattcacagatatttaatattatttcttacattattttgtttatt
ctgttctttgcatatgtgatgaacatcgaaagacaggactatagaagaag
tataaatgtaaccattcctgaacagtatacctgcaacaaaccttatactg
cgggaaataaggtagatgttgatataccaacatttaacagtttaaatact
gacgattattaa
Vaccinia Virus A29L gene sequence
Genbank-AY243312
Bases 140787-141704
SEQ ID NO 39:
atgcagcatccgcgggaagagaattcaatcgtcgttgaactcgaaccctc
attggctacatttatcaaacaaggatttaataatctcgtaaaatggccct
tgttaaacattggaatagttttgtctaatacatctaccgctgtcaatgag
gaatggctaactgcggtagagcatattcccaccatgaagatattttacaa
acatatacataagatacttactagagaaatggggtttttagtctatttga
aaagatcccaatctgaacgcgataattatataactttatacgattttgat
tattatattatagataaggatacaaattctgtaactatggtagataaacc
gaccgagttaaaggaaactttgttacatgtatttcaagaatatcgtttaa
agagttctcaaacaatagagcttatagcgtttagttcaggtacggtaata
aacgaagacatagtttcaaaattaacatttttagatgtggaggtatttaa
tagagaatataataatgttaaaactatcatagatccggattttgtattta
gatctccatttatagttatttctcctatgggtaaactaactttcttcgta
gaagtatattcgtggtttgattttaaatcgtgtttcaaagatattataga
tttcttagaaggtgctctaatagccaatattcataatcacatgattaagg
taggtaattgtgacgaaacagtatcgtcttataatccagagtctggaatg
ttgtttgttaatgacttaatgactatgaacatagtcaactttttcggatg
taattctaggttagaatcataccatcggttcgatatgacaaaagtagatg
tt
Vaccinia Virus G1L gene sequence
Genbank-AY243312
Bases 68977-70752
SEQ ID NO 40:
atgattgtcttaccgaataaagttcgtattttcatcaacgatcggatgaa
aaaggatatctacttgggaatttctaatttcggattcgagaatgatatag
atgaaatcttgggaattgctcacttgttggaacatctacttatatccttt
gattctactaattttttagcgaatgcttctacatctagaagttatatgag
tttttggtgtaaatccattaattcagcaacggaatcggacgcaatcagaa
cattagtttcgtggttcttttctaacggaaaactcaaagataatttttcc
ctttctagtatacgatttcacattaaagaattagaaaacgaatactattt
tagaaatgaagtattccattgtatggatatactaacgtttcttagcggag
gcgatttatataacggtgggagaatagacatgatagataatcttaatata
gttcgtgatatgctggtaaatagaatgcaaaggatatcgggatcgaatat
cgtaatttttgttaagagattaggacctggaacattggatttcttcaaac
agacatttgggtctttaccagcatgtccggagattattccttcgtctatt
ccagtaagtacaaacggtaaaatagttatgactccgtctccattttatac
agttatggtaaagattaatccaacattagataatattttagggattctgt
atttgtacgaaacttaccacttaatagactatgagactatcggcaaccag
ttatatttaacggtatcctttatcgatgaaactgaatacgagagctttct
tcgtggcgaggctatattacaaattagtcaatgtcaacgtattaatatga
attatagcgacgattatatgatgaacatctatttgaattttccttggcta
tcgcatgatttatatgattacattacacgtattaatgacgatagcaagtc
gatactaatatccttgacgaatgaaatatatgcatctataattaatagag
atatcatagttatttacccaaactttagtaaggccatgtgtaacactaga
gatacccaacaacatccgatagtagttcttgacgcaaccaatgatggact
gattaagaaaccttatagaagtatacccctaatgaagcgtctaacatcta
atgaaatatttatacgatacggagacgcgtctctcatggacatgataact
ttatcattgtctaaacaagatatatcattaaaaagaaatgccgaaggaat
acgtgtaaaacatagtttttcagctgatgatatacaggcaattatggaat
ctgattcgtttttaaagtatagtagatcaaaaccagctgcgatgtatcaa
tatatatttctatcattttttgctagtggtaattccatagatgacatatt
ggcaaatagagattctaccttagaattttctaaaagaactaaaagtaaaa
ttttgtttggtaggaataccaggtacgacgtcactgcaaaatctagtttt
gtatgtggtatagtacgaggtaaatcattggataaaacgtctctggttga
aatgatgtgggatctcaagaagaaaggattaatatattctatggaattta
ccaatctattgagtaagaataccttttatttgttcacatttactatctac
actgatgaagtatacgattatctaaacactaataaacttttttttgcaaa
atgtttagtcgtgtctacaaaaggagatgtagaaaatttttcatctctaa
aaaaagatgtggtcattagagtttga
Vaccinia Virus E1L gene sequence
Genbank-AY243312
Bases 44004-45443
SEQ ID NO 41:
atgaataggaatcctgatcagaatactcttcctaatattacattaaagat
tatagaaacctatttaggcagagtacctagtgtgaacgaatatcatatgt
taaaattacaagctagaaatattcaaaaaataactgtttttaacaaagac
atatttgtatctttagtaaaaaagaataaaaaaagatttttttccgatgt
taatacatctgcatcagaaataaaagatcgtatacttagctacttttcta
aacagactcaaacatataatataggtaaattatttacgattatagaacta
caatctgtattagtgaccacatacacggacatattaggagttcttactat
taaagctccaaatgtaatttcatctaaaatttcttataatgtaacatcaa
tggaagaattggcaagagatatgctaaattctatgaacgtcgcagtaata
gacaaggcaaaagtaatgggacgtcataatgtatcttccctagtcaaaaa
tgttaataagttgatggaagaatatcttagacgccataataaaagttgta
tatgttacggatcatattctctatatctaattaatccaaatatacggtac
ggcgatatagatattcttcagactaattctagaacttttcttatagattt
ggcctttctaataaaatttatcacgggaaataatattatattaagtaaaa
tcccatatcttagaaactatatggtgataaaagatgaaaacgataatcat
atcattgatagttttaatattcgccaggataccatgaacgtagttcctaa
aatctttatagataatatctatatagtggatccgacgtttcaactattga
acatgataaaaatgttttctcaaatagatagattggaagatctatccaaa
gatcctgaaaagtttaatgcgcgtatggcaaccatgctagaatacgttag
atatacacatggtatagtctttgatggtaagcgtaataatatgccgatga
aatgtatcatcgatgaaaataatcgcatagttactgtcactactaaagac
tattttagctttaaaaaatgtctagtgtatctagatgaaaatgtgttatc
gagtgatatattagatcttaacgccgacacatcgtgtgatttcgagagtg
ttacaaattctgtatatctaattcatgataatatcatgtatacatatttc
tcaaatactattctccttagtgataaggggaaggtacatgaaataagtgc
cagaggtttatgtgcacatatattgttgtatcagatgctgacatctggag
aatacaaacaatgtttatcggatctcttaaattcgatgatgaatagagat
aaaatacctatctattcacatactgaaagagataaaaaacctggacgaca
cggatttattaatatcgaaaaggatataattgtattttag
Vaccinia Virus D7R gene sequence
Genbank-AY243312
Bases 102613-103098
SEQ ID NO 42:
atgtcgagctttgttaccaatggataccttccagttacattggaaccaca
cgagctgacgttagacataaaaactaatattaggaatgccgtatataaga
cgtatctccatagagaaattagtggtaaaatggccaagaaaatagaaatt
cgtgaagacgtggaattacctctcggcgaaatagttaataattctgtagt
tataaacgttccgtgtgtaataacctacgcgtattatcacgttggggata
tagtcagaggaacattaaacatcgaagatgaatcaaatgtaactattcaa
tgtggagatttaatctgtaaactaagtagagattcgggtactgtatcatt
tagcgattcaaagtactgcttttttcgaaatggtaatgcgtatgacaatg
gcagcgaagtcactgccgttctaatggaggctcaacaaggtatcgaatct
agttttgtttttctcgcgaatatcgtcgactcataa
Vaccinia Virus D5R gene sequence
Genbank-AY243312
Bases 98275-100632
SEQ ID NO 43:
atggatgcggctattagaggtaatgatgttatctttgttcttaagactat
aggtgtcccgtcagcgtgcagacaaaatgaagatccaagatttgtagaag
catttaaatgcgacgagttagaaagatatattgagaataatccagaatgt
acactattcgaaagtcttagggatgaggaagcatactctatagtcagaat
tttcatggatgtagatttagacgcgtgtctagacgaaatagattatttaa
cggctattcaagattttattatcgaggtgtcaaactgtgtagctagattc
gcgtttacagaatgcggcgccattcatgaaaatgtaataaaatccatgag
atctaatttttcattgactaagtctacaaatagagataaaacaagttttc
atattatctttttagacacgtataccactatggatacattgatagctatg
aaacgaacactattagaattaagtagatcatctgaaaatccactaacaag
atcgatagacactgccgtatataggagaaaaacaactcttcgggttgtag
gtactaggaaaaatccaaattgcgacactattcatgtaatgcaaccaccg
catgataatatagaagattacctattcacttacgtggatatgaacaacaa
tagttattacttttctctacaacaacgattggaggatttagttcctgata
agttatgggaaccagggtttatttcattcgaagacgctataaaaagagtt
tcaaaaatattcattaattctataataaactttaatgatctcgatgaaaa
taattttacaacggtaccactggtcatagattacgtaacaccttgtgcat
tatgtaaaaaacgatcgcataaacatccgcatcaactatcgttggaaaat
ggtgctattagaatttacaaaactggtaatccacatagttgtaaagttaa
aattgttccgttggatggtaataaactgtttaatattgcacaaagaattt
tagacactaactctgttttattaaccgaacgaggagaccatatagtttgg
attaataattcatggaaatttaacagcgaagaacccttgataacaaaact
aattttgtcaataagacatcaactacctaaggaatattcaagcgaattac
tctgtccaagaaaacgaaagactgtagaagctaacatacgagacatgtta
gtagattcagtagagaccgatacctatccggataaacttccgtttaaaaa
tggtgtattggacctggtagacggaatgttttactctggagatgatgcta
aaaaatatacgtgtactgtatcaaccggatttaaatttgacgatacaaag
ttcgtcgaagacagtccagaaatggaagagttaatgaatatcattaacga
tatccaaccattaacggatgaaaataagaaaaatagagagctatatgaaa
aaacattatctagttgtttatgcggtgctaccaaaggatgtttaacattc
ttttttggagaaactgcaactggaaagtcgacaaccaaacgtttgttaaa
gtctgctatcggtgacctgtttgttgagacgggtcaaacaattttaacag
atgtattggataaaggacctaatccatttatcgctaacatgcatttgaaa
agatctgtattctgtagcgaactacctgattttgcctgtagtggatcaaa
gaaaattagatctgacaatattaaaaagttgacagaaccttgtgtcattg
gaagaccgtgtttctccaataaaattaataatagaaaccatgcgacaatc
attatcgatactaattacaaacctgtttttgataggatagataacgcatt
aatgagaagaattgccgtcgtgcgattcagaacacacttttctcaacctt
ctggtagagaggctgctgaaaataatgacgcgtacgataaagtcaaacta
ttagacgaggggttagatggtaaaatacaaaataatagatatagattcgc
atttctatacttgttggtgaaatggtacagaaaatatcatgttcctatta
tgaaactatatcctacacccgaagagattcctgactttgcattctatctc
aaaataggtactctgttagtatctagctctgtaaagcatattccattaat
gacggacctctccaaaaagggatatatattgtacgataatgtggtcactc
ttccgttgactactttccaacagaaaatatccaagtattttaattctaga
ctatttggacacgatatagagagcttcatcaatagacataagaaatttgc
caatgttagtgatgaatatctgcaatatatattcatagaggatatttcat
ctccgtaa
Vaccinia Virus A9L gene sequence
Genbank-AY243312
Bases 118842-119168
SEQ ID NO 44:
atgtcatgttatacagctatattaaaatctgtaggaggactggcgctatt
tcaagtagccaatggcgccatagatttatgtagacatttctttatgtatt
tttgtgaacaaaagctacgaccaaattcattttggttcgttgttgttaga
gccattgcgagcatgataatgtatttagtattaggtatagcattgctgta
tatttctgaacaagatgacaagaagaatactaataatgccaacactaata
atgatagtaatagtaataatagtaacaatgataaacgaaatgagtcgtct
ataaattctaactccagtcctaagtaa
Vaccinia Virus A56R gene sequence
Genbank-AY243312
Bases 162183-163127
SEQ ID NO 45:
atgacacgattaccaatacttttgttactaatatcattagtatacgctac
accttttcctcagacatctaaaaaaataggtgatgatgcaactctatcat
gtaatcgaaataatacaaatgactacgttgttatgagtgcttggtataag
gagcccaattccattattcttttagctgctaaaagcgacgtcttgtattt
tgataattataccaaggataaaatatcttacgactctccatacgatgatc
tagttacaactatcacaattaaatcattgactgctagagatgccggtact
tatgtatgtgcattctttatgacatcaactacaaatgacactgataaagt
agattatgaagaatactccacagagttgattgtaaatacagatagtgaat
cgactatagacataatactatctggatctacacattcaccggaaactagt
tctaagaaacctgattatatagataattctaattgctcgtcggtattcga
aatcgcgactccggaaccaattactgataatgtagaagatcatacagaca
ccgtcacatacactagtgatagcattaatacagtaagtgcatcatctgga
gaatccacaacagacgagactccggaaccaattactgataaagaagatca
tacagttacagacactgtctcatacactacagtaagtacatcatctggaa
ttgtcactactaaatcaaccaccgatgatgcggatctttatgatacgtac
aatgataatgatacagtaccaccaactactgtaggcggtagtacaacctc
tattagcaattataaaaccaaggactttgtagaaatatttggtattaccg
cattaattatattgtcggccgtggcaattttctgtattacatattatata
tataataaacgttcacgtaaatacaaaacagagaacaaagtctag
Vaccinia Virus L4R gene sequence
Genbank-AY243312
Bases 79139-79894
SEQ ID NO 46:
atgagtctactgctagaaaacctcatcgaagaagataccatattttttgc
aggaagtatatctgagtatgatgatttacaaatggttattgccggcgcaa
aatccaaatttccaagatctatgctttctatttttaatatagtacctaga
acgatgtcaaaatatgagttggagttgattcataacgaaaatatcacagg
agcaatgtttaccacaatgtataatataagaaacaatttgggtctaggag
atgataaactaactattgaagccattgaaaactatttcttggatcctaac
aatgaagttatgcctcttattattaataatacggatatgactgccgtcat
tcctaaaaaaagtggtaggagaaagaataagaacatggttatcttccgtc
aaggatcatcacctatcttgtgtattttcgaaactcgtaaaaagattaat
atttataaagaaaatatggaatccgcgtcgactgagtatacacctatcgg
agacaacaaggctttgatatctaaatatgcgggaattaatatcctaaatg
tgtattctccttccacatccataagattgaatgccatttacggattcacc
aataaaaataaactagagaaacttagtactaataaggaactagaatcgta
tagttctagccctcttcaagaacccattaggttaaatgattttctgggac
tattggaatgtgttaaaaagaatattcctctaacagatattccgacaaag
gattga
Vaccinia Virus G4L gene sequence
Genbank-AY243312
Bases 71710-72084
SEQ ID NO 47:
atggccgaggaatttgtacaacaaaggttggccaataacaaagtgacaat
ttttgtcaagtatacatgtcctttttgtagaaatgcactggatattctaa
ataagtttagtttcaaaagaggagcgtatgaaattgtcgatattaaagaa
tttaaacccgaaaatgaattgcgtgactattttgaacaaattactggtgg
tagaactgttcctagaatcttttttgggaaaacttctattggtggatata
gcgacctgttggaaatagacaacatggacgcattgggtgatattctatca
tctattggggtattgagaacttgttga
Vaccinia Virus E9L gene sequence
Genbank-AY243312
Bases 53636-56656
SEQ ID NO 48:
atggatgttcggtgcattaattggtttgaaagtcacggtgaaaacagatt
tttatatctgaaatccagatgtcgaaatggtgagaccgtatttatacgat
ttcctcattacttttattacgtagtgacggacgaaatatatcagtcattg
tctcctcctccatttaatgcgaggccgttgggaaagatgagaactataga
cattgacgagacaataagttataatctagatattaaagatagaaaatgct
ccgtcgcagatatgtggttgatagaagagccaaagaaacgcagcatacaa
aatgccaccatggatgaatttctcaatattagttggttttatatttctaa
cgggatatctccagacggatgttactcgttggacgagcaatatttgacaa
agattaacaatggatgttatcattgtgacgatccacgtaactgtttcgct
aaaaaaatacctagattcgatatcccaagatcgtacttatttctagatat
agagtgtcacttcgataagaagtttccttctgtatttattaacccaatct
cgcatacaagttactgttatatcgatttaagtggtaaacgattattgttt
acgctcattaatgaagagatgttaacggaacaggaaatacaagaagccgt
cgatagaggatgtttgaggatacagtcactaatggaaatggattacgaac
gagaactagttttatgttctgaaatagttttgttacgaatagctaaacaa
ttgttggaactaacgttcgactatgtcgttacctttaacggacataactt
tgatctgagatatattactaatcgtctagagttattaacaggagagaaga
ttatctttagatctccggacaaaaaggaagctgtacatctctgtatttat
gagagaaatcagtctagtcataagggagtaggcggcatggccaatactac
gtttcacgttaataacaataatggaactatatttttcgatctatattcat
tcattcaaaaatctgaaaaattggattcgtacaaattggattctatatcc
aagaacgcgttcagttgcatgggtaaagtattaaatagaggagttagaga
aatgacgttcatcggtgacgatactacggacgcgaaaggcaaagccgctg
catttgcaaaggttttaaccacaggtaattatgtgactgttgatgaggat
attatatgtaaagtaattcgtaaagatatttgggaaaatggatttaaagt
cgtactattatgtcctactttacctaatgatacatataaattatctttcg
gaaaggatgacgttgatttagctcagatgtataaggattataatctaaac
atagctttagatatggctagatactgtattcatgatgcttgtttgtgtca
gtatttgtgggagtattatggagtagaaacaaaaacagacgcgggtgcgt
caacatatgtgcttcctcaatccatggtattcgaatatagagcgagtaca
gtcatcaagggtccactgttaaagctattgttggaaactaaaactatctt
agttagatcagaaacaaaacaaaagtttccttatgaaggcggtaaggtat
ttgctccaaaacaaaaaatgtttagtaataatgtattaatctttgattat
aacagtctgtatcctaatgtgtgtatctttggaaatctatctccggaaac
attagtcggtgtcgttgttagtaccaatagattggaagaagaaataaata
atcagctcttgcttcagaaatatccacctcctagatatattacggttcat
tgtgaacctagactaccgaacctcatctctgaaatagcaattttcgatag
atcgatagaaggaaccattcctagactattaagaacatttttggcagaga
gagccagatataaaaagatgttaaaacaggctaccagttcaactgaaaag
gccatctatgattccatgcaatatacgtacaagatagtagccaactcagt
atatggtctgatgggatttagaaatagtgctctatactcatacgcttcgg
ctaagagttgcacatccataggacgtagaatgatcttgtatctagaatcg
gtactaaatggagcagagttatctaacggtatgttacggtttgccaatcc
attaagtaatccattttatatggacgatagagatattaatccgattgtga
aaacatcgttgcctatagattacagatttcgttttcgtagcgtgtatgga
gataccgactccgtgtttacagagatagacagtcaagatgtagataagtc
catagaaatagcaaaggagttagaacgactgattaataatagagtattgt
ttaataattttaaaatagagtttgaggcggtatataagaatctgattatg
caatcgaagaagaaatatacaacgatgaaatactcggcatcgtcgaattc
aaaatctgtacctgagagaattaataaaggtactagtgaaactagaagag
atgtttccaagtttcataagaatatgattaagacatacaagaccagactg
tctgagatgttgtctgaaggacggatgaattctaatcaggtatgtataga
tattctccgttctttagaaacagatttacgatccgaatttgatagtagat
cgtctcctctagaattatttatgttgagtcgaatgcatcactcaaattat
aaatccgcagataaccctaatatgtatttggttactgaatataataaaaa
taatccagaaactatagaacttggagaacgatattattttgcatatattt
gtccggctaatgtaccatggaccaaaaaacttgtaaatattaaaacatat
gaaacaattatcgatagaagttttaaactcggcagtgatcaaagaatatt
ttacgaagtttactttaaacgattgacgtccgaaatagtcaatctattgg
ataataaagttttatgcatctcattctttgaaagaatgtttggttcaaaa
cctacattttacgaagcataa

1-42. (canceled)

43. An isolated RNA molecule that inhibits expression of a poxvirus gene chosen from A17L, A29L, G1L, E1L, D7R, D5R, A9L, A56R, L4R, G4L, E9L, and F10L, wherein the isolated RNA molecule comprises 19 to 27 nucleotides sufficiently complementary to SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or the reverse complement of SEQ ID NO:37, respectively, to inhibit expression of the poxvirus gene.

44. The isolated RNA molecule of claim 43 wherein the isolated RNA molecule is double stranded.

45. The isolated RNA molecule of claim 43 wherein the isolated RNA molecule is an siRNA or an shRNA.

46. The isolated RNA molecule of claim 43 wherein the 19 to 27 nucleotides of the isolated RNA molecule are complementary to SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or the reverse complement of SEQ ID NO:37.

47. The isolated RNA molecule of claim 43 wherein the isolated RNA molecule is chemically modified.

48. The isolated RNA molecule of claim 47 wherein the isolated RNA molecule comprises at least one backbone-modified ribonucleotide

49. The isolated RNA molecule of claim 43 wherein the isolated RNA molecule comprises at least one ribonucleotide modified at the base portion, the sugar portion, or the phosphate portion.

50. The isolated RNA molecule of claim 49 wherein the isolated RNA molecule comprises at least one sugar-modified ribonucleotide, wherein the 2′-OH group of the sugar-modified ribonucleotide is replaced by a group chosen from H, OR, R, halo, SH, SR, NH2, NHR, N(R)₂, and CN, wherein R is C1-C6 alkyl, alkenyl, or alkynyl, and wherein halo is F, Cl, Br, or I.

51. The isolated RNA molecule of claim 44 wherein one strand of the double stranded RNA molecule comprises a 3′ overhang of from 1 to 6 ribonucleotides.

52. The isolated RNA molecule of claim 43 wherein the isolated RNA molecule comprises non-nucleotide material.

53. The isolated RNA molecule of claim 51 wherein the non-nucleotide material comprises cholesterol.

54. A vector encoding the isolated RNA molecule of claim 43.

55. A cell comprising the vector of claim 54.

56. A composition comprising the isolated RNA molecule of claim 43 and a pharmaceutically acceptable carrier.

57. A method for inhibiting poxviral replication comprising administering a therapeutically effective amount of the composition of claim 56 to a subject in need thereof.

58. A kit comprising a container and the isolated RNA molecule of claim 43.
